(19) United States
(12) Patent Application Publication
(10) Pub. No.: US 2006/0128792 A1
(43) Pub. Date: Jun. 15, 2006

(54) Deguelin as a chemopreventive agent for lung cancer

(76) Inventor: Ho-Young Lee, Houston, TX (US)

Correspondence Address: FULBRIGHT & JAWORSKI L.L.P., 600 CONGRESS AVE., SUITE 2400, AUSTIN, TX, 78701, US

(21) Appl. No.: 10/531,140

(22) PCT Filed: Oct. 10, 2003
(86) PCT No.: PCT/US03/32263

§ 371 Date: Dec. 22, 2005

Related U.S. Application Data
(60) Provisional application No. 60/417,966, filed on Oct. 11, 2002.

(51) Int. Cl. A61K 31/353; A61K 31/337; A61K 31/704; A61N 5/00

(52) U.S. Cl. 514/453; 514/449; 514/34; 600/1

(57) ABSTRACT

The present invention provides the chemopreventive agent deguelin, a natural product isolated from Mundulea serica (Leguminosae), and derivatives thereof, for use in combination with a second agent for inhibiting growth premalignant and malignant lung cancer cells by causing G2/M arrest and apoptosis. Thus, the present invention provides deguelin-based combination therapies for the treatment and prevention of lung cancer. The second agent of the present invention may, in particular, be an inhibitor of the P13K, MAPK or JNK signaling pathways, or a chemotherapeutic agent, or radiotherapeutic agent.

### BACKGROUND OF THE INVENTION

1. Field of the Invention

The present invention relates generally to the fields of cancer biology and cancer therapy. More particularly, it concerns the use of deguelin and derivatives thereof in combination with a second agent in the treatment and prevention of lung cancer disease.

2. Description of Related Art

In the United States, lung cancer leads all other cancers in both incidence and mortality rate (Khuri et al., 2001). Lung cancer is the primary cause of cancer death among both men and women in the U.S., and worldwide. The five-year survival rate among all lung cancer patients in the U.S., regardless of the stage of disease at diagnosis, is only 13%. This contrasts with a five-year survival rate of 46% among cases detected while the disease is still localized. However, only 16% of lung cancers are discovered before the disease has spread.

Early detection is difficult since clinical symptoms are often not seen until the disease has reached an advanced stage. Currently, diagnosis is aided by the use of chest x-rays, analysis of the type of cells contained in sputum and fiberoptic examination of the bronchial passages. Treatment regimens are determined by the type and stage of the cancer, and include surgery, radiation therapy and/or chemotherapy. Despite recent advances in radiotherapy and chemotherapy modalities, the severe morbidity of lung cancer and the poor 5-year survival rates have not improved (Khuri et al., 2001).

Thus, cancer chemoprevention is a logical and obvious strategy to help alleviate this disease (Watenberg, 1992; Kelloff et al., 1994). Chemoprevention targets the multistep process of carcinogenesis with chemical agents that delay, reverse, or block cancer development (Lee et al., 2001). The exposure of aerodigestive tract epithelium to carcinogenic and tumor-promoting agents often leads to histologic changes over large areas of the tissue, resulting in a field cancerization with potential multifocal unsynchronized, premalignant and primary malignant lesions (Lotan, 1996). One of the major needs in cancer prevention is the development of new, effective and safe chemopreventive agents, especially agents targeted at mechanisms known to be involved in the process of carcinogenesis.

Carcinogenesis is a multistep process that is driven by various genetic defects (Ahmadian et al., 1999). Among these defects is the proto-oncogene ras, which participates in the early phase of tumor development (Kinzler et al., 1996). Ras mutations have been found in a wide variety of human malignancies including lung cancer. Oncogenic mutations in ras result in activation of downstream signaling proteins, including the Raf/MEK/ERK (Robinson et al., 1997) and the PI3K/Akt pathway (Rodriguez-Viciana et al., 1997), regulating cell proliferation, viability, and differentiation in both normal and transformed cell types. PI3K/Akt in particular has demonstrated a clear role in oncogenic transformation (Di Cristofano et al., 2000).

Since clinical studies have showed that chemoprevention of aerodigestive tract cancer is feasible and effective (Hong et al, 1997; Benner et al., 1992; Lee et al., 2001), there has been a shift of interest toward the strategies of early detection and effective chemoprevention, and much effort has been devoted to the discovery and development of new chemopreventive agents. Retinoids, antihormones, antioxidants, biologic modifiers, nonsteroidal anti-inflammatory agents, trace elements, and ornithine decarboxylase (ODC) inhibitors are examples of chemopreventive agents that have been used successfully in either animal experimental carcinogenesis models or clinical trials (Watenberg, 1992; Kelloff et al., 1994). However, undesirable side effects or resistance of lung cancer cells to these agents limit their long-term clinical use as chemopreventive agents. Therefore, the present invention provides novel agents to effectively treat and prevent lung cancer with minimal toxicity.

### SUMMARY OF THE INVENTION

The present invention is directed to a chemopreventive therapy for lung cancer disease and overcomes the deficiencies in the art of current therapies such as radiotherapy and chemotherapy in combating lung cancer disease. The present invention addresses the need for more desirable chemopreventive agents to overcome toxicity, side effects or resistance offered by current chemopreventive agents in the treatment and prevention of lung cancer disease. The present invention provides a chemopreventive strategy for the treatment and prevention of lung cancer with minimal toxicity, side effects or resistance.

Thus, the present invention provides a method of inhibiting growth in a lung cancer cell comprising contacting the cell with a therapeutically effective amount of deguelin or a derivative thereof in combination with a second agent. It is contemplated in some embodiments of the invention that the second agent is an inhibitor of the signal transduction pathway involved in proliferation and apoptosis. Such an inhibitor includes, but is not limited to, a PI3K inhibitor, a MAPK inhibitor or a JNK inhibitor. It is further contemplated that in other embodiments of the invention, the second agent may be a chemotherapeutic agent such as taxol or doxorubicin or a radiotherapeutic agent.

In particular embodiments, the present invention provides a deguelin derivative in combination with a second agent for inhibiting lung cancer disease. Deguelin derivatives contemplated by the present invention include but are not limited to: 6a,2a-dehydrorotenone, methoxyrot-2′-enoic acid, tephrosin, 7S-hydroxydeguelin, rotenone, 7a,13a-dehydrodeguelin, 12-hydroxyrotenone, 12,12a-dehydrorotenone, isorotenone, 4-chlororot-2′-enoic acid, 1,2-dihydrodeguelin, 2-phenylselenyl-1,2-dihydrodeguelin, or bromorot-2′-enoic acid.

In other embodiments, the invention, further comprises a method of inducing apoptosis in lung cancer cells comprising contacting the cell with a therapeutically effective amount of deguelin or a derivative thereof in combination with a second agent.

In further embodiments of the present invention, the lung cancer cell is a cell culture or a tissue culture. In yet a further embodiment of the invention, the lung cancer cell is in a mammal such as a human. In still further embodiments of the invention, the lung cancer cell is a premalignant lung cancer cell, a malignant lung cancer cell, or a metastatic lung cancer cell. In further embodiments of the invention, the cancer to be treated with deguelin or derivatives thereof include, but are not limited to, breast cancer, prostate cancer, ovarian cancer, or head & neck cancer.

In particular embodiments of the invention, the lung cancer cell is a non-small cell lung cancer cell, a small cell lung cancer cell, or a rare lung cancer cell. In a further embodiment, the non-small cell lung cancer is a squamous cell carcinoma, an adenocarcinoma or a large cell carcinoma. In still yet a further embodiment, the small cell lung cancer is a lymphocytic small cell lung cancer, a intermediate small cell lung cancer or a combined small cell lung cancer.

In a particular embodiment, the combined small cell lung cancer further comprises small cell lung cancer and squamous cell carcinoma. In a further embodiment, the combined small cell lung cancer further comprises small cell lung cancer and adenocarcinoma. In still a further embodiment, the rare lung cancer cell is a adenoid cystic carcinoma, a mesothelioma, a hamartoma, a lymphoma or a sarcoma. In some embodiments, the lung cancer cell is a carcinoid tumor cell. Any type of lung cancer cell is contemplated within the scope of the present invention.

The present invention further provides a method for treating or preventing lung cancer in a subject comprising providing to the subject a therapeutically effective amount of deguelin or derivative thereof, in combination with a second agent. In a particular embodiment, the invention further provides a method of inducing apoptosis in a lung cancer cell. Derivatives of degulein contemplated for use in the present invention in combination with a second agent for treating and preventing lung cancer include but are not limited to: 6a,2a-dehydrorotenone, methoxyrot-2′-enoic acid, tephrosin, 7S-hydroxydeguelin, rotenone, 7a,13a-dehydrodeguelin, is 12-hydroxyrotenone, 12,12a-dehydrorotenone, isorotenone, 4-chlororot-2′-enoic acid, 1,2-dihydrodeguelin, 2-phenylselenyl-1,2-dihydrodeguelin, or bromorot-2′-enoic acid. In yet a further embodiment, the second agent contemplated for use in the present invention for treating or preventing lung cancer in a subject is an inhibitor of the signal transduction pathways involved in proliferation and apoptosis. Such an inhibitor include but is not limited to a PI3K, MAPK or JNK inhibitor. Other second agents contemplated in the present invention include, but are not limited to chemotherapeutic agents such as taxol or doxorubicin, or radiotherapeutic agents.

In another embodiment of the present invention, a therapeutically effective amount of deguelin or a derivative thereof is provided to a subject before the second agent, after the second agent or at the same time as the second agent for treating or preventing lung cancer in the subject. In further embodiments of the invention, deguelin or a derivative thereof is provided once, or more than once. In a particular embodiment of the invention, a therapeutically effective amount of deguelin or a derivative thereof is provided to a subject intratumorally, intravenously, intraperitoneally, intramuscularly, orally, or by inhalation

In another embodiment of the invention, the second agent is provided once or more than once to the subject. In yet another embodiment of the invention, a therapeutically effective amount of the second agent is provided to a subject intratumorally, intravenously, intraperitoneally, intramuscularly, orally, or by inhalation.

In a further embodiment, deguelin or a derivative thereof in combination with a second agent is provided once or more than once to a subject.

In some embodiments, the present invention provides a method for treating or preventing lung cancer in a subject comprising providing to the subject a therapeutically effective amount of deguelin or derivative thereof in combination with a second agent and an additional therapeutic modality. Such additional therapeutic modalities include but are not limited to photodynamic therapy or surgery.

In yet another embodiment, the present invention provides a method for assaying for the inhibition of lung cancer cell growth comprising: (a) providing a lung cancer cell sample; (b) contacting the cell with an effective amount of deguelin or derivative thereof and a second agent; (c) analyzing the cell for growth inhibition; and, (d) comparing the inhibition of the cell growth in step (c) with the inhibition of a lung cancer cell in the absence of deguelin or derivative thereof and a second agent, wherein the difference in growth inhibition represents the growth inhibitory effect of deguelin or derivative thereof and a second agent.

In a further embodiment, the invention contemplates analyzing growth inhibition in a lung cancer cell by MTT assay. In yet a further embodiment, the invention contemplates analyzing a lung cancer cell for induction of apoptosis by FACS. In still yet a further embodiment, the present invention contemplates analyzing a lung cancer cell for inhibition of Akt activity by PI3K assay.

In still yet other embodiments, the present invention contemplates a pharmaceutical composition comprising deguelin derivatives and a second agent. Such deguelin derivatives contemplated by the invention are: 6a,2a-dehydrorotenone, methoxyrot-2′-enoic acid, tephrosin, 7S-hydroxydeguelin, rotenone, 7a,13a-dehydrodeguelin, is 12-hydroxyrotenone, 12,12a-dehydrorotenone, isorotenone, 4-chlororot-2′-enoic acid, 1,2-dihydrodeguelin, 2-phenylselenyl-1,2-dihydrodeguelin, or bromorot-2′-enoic acid, but are not limited to such derivatives.

In other embodiments, the present invention contemplates a pharmaceutical composition comprising deguelin and a second agent wherein the second agent is an inhibitor of the signal transduction pathways involved in proliferation and apoptosis. Such an inhibitor include but is not limited to a PI3K, MAPK or JNK inhibitor. The second agent may also be a chemotherapuetic agent or a radiotherapeutic agent. The chemotherapuetic agent may include, but not be limited to, taxol or doxorubicin.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Structure of deguelin.

FIGS. 2A-2B—Comparison of responses of normal (NHBE), premalignant (1799, 1198), and malignant (1170) HBE cells to growth-inhibitory effects of deguelin. (FIG. 2A) Cells were seeded in 96-well culture plates (2000-5000 cells/well) and treated with indicated concentrations of deguelin for 1, 2, or 3 days. Cell viability was measured by MTT assay. Results are expressed relative to the cell density of DMSO-treated cells at day 1. Each value is the mean (±SD) of six identical wells. (FIG. 2B) Effects of deguelin on cell cycle of 1799 cells and NHBE cells. 1799 cells or NHBE cells exposed to 0.1% DMSO (con) or to indicated concentrations of deguelin for either 3 days (NHBE cells) or 1, 2, or 3 days (1799 cells) were analyzed for DNA content (propidium iodide uptake).

FIG. 3—Evidence of apoptosis in 1799 cells treated with deguelin. 1799 cells were treated with 10⁻⁹ M to 10⁻⁷ M of deguelin for 3 days. Following harvest, fixation, and permeabilization of the cells, TUNEL analysis was performed using an APO-BRDU staining kit (Phoenix Flow Systems, San Diego, Calif.). All values presented are the percentage of cells as determined by light scatter. The percentage of dead cells was determined by FACS analysis of propidium iodide-stained nuclei.

FIGS. 4A-4B—Protection of premalignant HBE cells from deguelin-induced cell death by activated Akt. (FIG. 4A) Viability of 1799 infected with Ad5CMV-Myr.Akt-HA in response to deguelin treatment. 1799 HBE cells were either uninfected (con) or infected with either 5×10³ particles per cell (p/cell) of Ad5CMV or 1×10³ or 5×10³ p/cell of Ad5CMV-MyrAkt-HA in the KSFM for 1 day, and then treated with 10⁻⁷ M or 10⁻⁶ M deguelin for 2 days. Results are expressed relative to the cell density of untreated cells. Each value is the mean (±SD) of five identical wells. (FIG. 4B) Activated Akt protects 1799 cells from deguelin-induced apoptosis. Induction of apoptosis by 10⁻⁷ M of deguelin in 1799 cells that were uninfected (con) or infected with indicated titers (p/cell) of either Ad5CMV or Ad5CMV-Myr.Akt-HA was analyzed by flow cytometry.

FIG. 5.—Flow cytometry analysis of deguelin on HBE cells. FACS analysis was performed on H1299 and squamous HBE cells untreated (0 d) or treated with the 10⁻⁷-M deguelin for 1, 2, or 3 days. All values presented are percentages of cells determined by light scatter.

FIGS. 6A-6B.—Growth-inhibitory effects of deguelin on NSCLC cell proliferation. (FIG. 6A) Cells were seeded in 96-well culture plates (2000-4000 cells/well) and treated with indicated concentrations of deguelin for 1, 2, or 3 days. Cell viability was measured by MTT assay. Results are expressed relative to the cell density of DMSO-treated cells at day 1. Each value is the mean (±SD) of six identical wells. (FIG. 6B) The growth inhibitory effects of deguelin on normal and squamous HBE cells were compared with the effect on NSCLC cells.

FIG. 7.—Growth-inhibitory effects of deguelin derivatives on NSCLC cell proliferation. Cells were seeded in 96-well culture plates (2000-4000 cells/well) and treated with indicated concentrations of deguelin for 1, 2, or 3 days. Cell viability was measured by MTT assay. Results are expressed relative to the cell density of DMSO-treated cells at day 1. Each value is the mean (±SD) of six identical wells.

FIG. 8.—Deguelin inhibits cell proliferation in vivo. Growth of NSCLC xenografts is inhibited by treatment of deguelin. The results are expressed as the mean (±SD) tumor volume (calculated from at least 5 mice) relative to the initial volume.

FIGS. 9A-9D.—Anti-angiogenic activity of deguelin. (FIG. 9A) CAMs after incubation with Thermanox coverslips containing vehicle (Con), deguelin (1 or 5 nM) or RA (1 μg) as a positive control or for 48 h (circle indicate the placement of coverslip). (FIG. 9B) The anti-angiogenic effect of deguelin was evaluated by calculating the percentage of positive eggs. □ empty coverslip as control; ▪ RA 1 μg/egg; ▪ deguelin 1 or 5 nmole/egg. Each value represents the mean±SE. (FIG. 9C) Appearance of matrigel from mice. The effects of deguelin on bFGF-induced angiogenesis in vivo was analyzed by matrigel plug assay using nude mouse. Matrigel alone (Con)—negative control, 100 ng/ml bFGF and 72 units/ml heparin in a vehicle of 0.1% BSA/PBS (bFGF)—positive control, 5 niole deguelin alone, and 5 nmole deguelin plus bFGF were included. (FIG. 9D) Proliferation of HUVEC cells treated with indicated doses of deguelin for 3 days was analyzed by MTT assay. Bars, means±SD of a representative experiment done in triplicate from five independently performed experiments for each cell line.

FIG. 10. Deguelin sensitizes cancer cells to chemotherapeutic agents. The indicated doses of paclitaxel(taxol), doxorubicin, or 4Gy of irradiation (Rad) were added for 1 day before MTT analysis. Results are expressed relative to the density of untreated cells. Bars, means±SD of a representative experiment done in six identical wells from five independently performed experiments.

FIG. 11. Deguelin inhibits cell growth in prostate, breast, head & neck and ovarian cancer cell lines

### DESCRIPTION OF ILLUSTRATIVE EMBODIMENTS

Lung cancer is the primary cause of cancer death among both men and women worldwide. Despite recent advances in radiotherapy and chemotherapy modalities, the severe morbidity of lung cancer and the poor 5-year survival rates have not improved. Cancer chemoprevention provides and obvious strategy in overcoming the deficiencies in alleviating this disease.

### I. THE PRESENT INVENTION

The present invention concerns the use of the chemopreventive agent deguelin, a natural product isolated from Mundulea serica (Leguminosae). In particular, the present invention provides a method for treating and preventing lung cancer employing deguelin in combination with a second agent such as, but not limited to, inhibitors of the signal transduction pathways involved in the cell proliferation and apoptosis. The present invention also employs the use of derivatives of deguelin in combination with a second agent. The present invention contemplates as a second agent PI3K inhibitors, MAPK inhibitors and JNK inhibitors. In some embodiments, the present invention contemplates chemotherapeutic agents such as taxol or doxorubicin as a second agent. The present invention also contemplates radiotherapeutic agents as a second agent.

The present invention provides evidence for the first time that the Akt activity is consititutively active in premalignant HBE cell line, and that deguelin acts through this pathway. Thus, this provides an opportunity for the use of deguelin, in combination with a second agent, as a therapeutic or chemopreventive combination therapy against lung cancer. Deguelin (a) blocks proliferation of premalignant and malignant “BE cells through induction of the apoptosis; (b) is active at nanomolar levels and has no cytotoxicity on HBE cells, showing its therapeutic efficacy; and (c) selectively blocks Akt activity in either a PI3K-dependent or -independent manner, thereby attenuating the activity of a major antiapoptotic pathway. Conversely, overexpression of constitutively active Akt protected cells from deguelin-mediated apoptosis.

The role of deguelin as an inhibitor of Akt activation also has particular clinical implications where constitutive activation of Akt occurs at a high frequency (e.g., NSCLC; Yano et al., 1998). It has been reported that the manipulation of Akt activity alters the sensitivity of NSCLC cells to chemotherapy and irradiation and that addition of a PI3K inhibitor or transfection of kinase-dead Akt into cells with high levels of Akt activity causes dramatic sensitization to these treatments (Brognard et al., 2001). Therefore, targeting Akt using deguelin can enhance the efficacy of chemotherapy and radiation therapy and increase the apoptotic potential of NSCLC cells.

The results presented herein demonstrate that deguelin inhibits premalignant and malignant HBE cell proliferation without a detectable cytotoxicity on normal HBE cells. Presumably, this occurs as a result of the ability of deguelin to diminish the signal transduction pathway involving PI3K and Akt, which may explain its potency and specificity. Thus, the present invention provides for the use of deguelin in combination with a second agent, such as a inhibitor of the signal transduction pathway, as a novel drug. The specific sensitivity of 1799, squamous HBE cells and NSCLC cells to deguelin raises the possibility of its potential to be used in the clinic as a chemopreventive agent for the early stages of lung carcinogenesis as well as a therapeutic agent against lung cancer.

### II. DEGUELIN AND DERIVATIVES THEREOF

Deguelin belongs to the family of rotenone compounds. Rotenone, deguelin and related compounds (rotenoids) are the active ingredients of botanical insecticides used for at least 150 years to control crop pests (Negherbohn, 1959; Fukami et al., 1971). They have been used even longer as fish poisons by native tribes to obtain food (Negherbohn, 1959; Fukami et al., 1971) and more recently in fish management to achieve the desired balance of species (e.g., the 1997 treatment of Lake Davis in California; California Dept. Fish and Game, 1997). The acute toxicity of rotenone to insects, fish, and mammals is attributable to inhibition of NADH:ubiquinone oxidoreductase activity as the primary target (Fukami and Wilkinson, 1976; Hollingworth and Ahamrnmadsahib, 1995).

Rotenoids are known not only as toxicants, but also as candidate anticancer agents based on three observations: (a) dietary rotenone reduces the background incidence of liver tumors in mice (Cunningham et al., 1995) and mammary tumors in rats (Hansen et al., 1965); (b) prevents cell proliferation induced by a peroxisome proliferator in mouse liver (Cunningham et al., 1995); and (c) deguelin and three of its derivatives inhibit phorbol ester-induced ornithine decarboxylase (ODC) activity as a measure of cancer chemopreventive potency (Gerhaiuser et al., 1995; Luyengi et al., 1994). The commercial rotenone-containing botanicals or extracts thereof are complex mixtures of rotenoids and other natural products that provide the opportunity for action on multiple biochemical targets. It has been hypothesized that rotenone and other rotenoids inhibit NADH:ubiquinone oxidoreductase and induced ODC activities by totally different mechanisms. An alternative hypothesis is that the inhibition of NADH:ubiquinone oxidoreductase activity is coupled to the cancer chemopreventive action (Figueras and Gosalvez, 1973; Gosalvez et al., 1976) and to the lowering of induced ODC activity (Gerhauser et al., 1996; Rowlands and Casida, 1997) so the same primary target may be involved. A study by Rowlands and Casida (1997) with rotenone and deguelin led to the proposal that inhibition of NADH:ubiquinone oxidoreductase activity blocks multiple signal transduction pathways, possibly modulated by reactive oxygen species, that regulate ODC activity.

A. Deguelin Derivatives

Derivatives of deguelin are known in the art and have been shown to be involved in regulating activity of molecules such as ODC and to play a role in cancer prevention. These derivatives include but are not limited to: tephrosin, (−)-13 hydroxytephrosin, and (−)-13α hydroxydegulin which have been found to inhibit orinthine decarboxylase (ODC) activity induced by 12-O-tetradecanoylphorbol 13-acetate (TPA), in mouse epidermal cancer cells. Other derivatives of deguelin contemplated in the present invention are 6a,2a-dehydrorotenone, methoxyrot-2′-enoic acid, 7S-hydroxydeguelin, 7a,13a-dehydrodeguelin, 12-hydroxyrotenone, 12,12a-dehydrorotenone, isorotenone, 4-chlororot-2′-enoic acid, 1,2-dihydrodeguelin, 2-phenylselenyl-1,2-dihydrodeguelin, and bromorot-2′-enoic acid.

### III. INHIBITORS OF PI3K, MAPK, JNK

The present invention contemplates the use of a second agent in combination with deguelin or derivatives thereof as a lung cancer therapy. In particular the present invention contemplates inhibitors of the PI3K, MAPK and JNK signaling pathways as the second agent in combination with deguelin or derivatives thereof for treating lung cancer.

A. PI3K Inhibitors

PI3K has an active role in oncogenic transformation (Chang et al., 1997). PI3K also affects many biologic functions, such as cell survival, apoptosis, and glucose transport (Toker et al., 1997; Vanhaesbrock et al., 1997). Recent findings further support the concept that PI3K is involved in the development of cancer. Specifically, PIK3CA, encoding p110α, has been amplified in human ovarian cancer cell lines (Shayesteh et al., 1999), and an oncogenic mutant of p85 that can transform mammalian fibroblasts in collaboration with the v-raf oncogene has been isolated (Jimenez et al., 1998). In addition, a partially transformed phenotype in mammalian fibroblasts transfected with constitutively active form of p110α has been demonstrated (Klippel et al., 1998). The tumor suppressor protein PTEN, which dephosphorylates the D3-lipid product of PI3K, phosphatidylinositol 3,4,5-triphosphate, interferes with potentially oncogenic signals emanating from PI3K (Maehama et al., 1999; Cantley et al., 1999).

The transforming activity of PI3K is correlated with its ability to induce activating phosphorylation in Akt protein kinase (also called protein kinase B (PKB)). Akt phosphorylates a number of proapoptotic and antiapoptotic proteins, including the Bcl-2 family member BAD, caspase-9, cyclic AMP-response element-binding protein, IkappaB kinase alpha (IKKα), and forkhead transcription factor-1 (Di Cristofano et al., 2000). It has been demonstrated that Akt is an important and probably essential downstream component of the oncogenic signal from PI3K (Di Cristofano et al., 2000; Toker et al., 1997; Vanhaesbrock et al., 1997; Chang et al., 1997; Shayesteh et al., 1999; Jimenez et al., 1998; Klippel et al., 1998), and thus compounds that inhibit PI3K/Akt activity are of particular interest.

The present invention therefore contemplates the use of PI3K inhibitors in combination with deguelin or a derivative thereof as a lung cancer therapy. Phosphatidylinositol 3-kinase inhibitors are well know to those of skill in the art, and have been crucial in deciphering the roles of PI3Ks in cellular processes. Such inhibitors that are contemplated for use in the present invention include, but are not limited to, LY294002 and wortmannin which are both potent and specific PI3K inhibitors. LY294002, a synthetic compound that was designed as a PI3K inhibitor based on the flavonoid quercetin (Vlahos et al., 1994), was shown to inhibit phosphatidylinositol 3-kinase inhibitor by competing for phosphatidylinositol 3-kinase binding of ATP. LY294002 was shown to act in vivo as a highly selective inhibitor of phosphatidylinositol 3 (PI3) kinase (Vlahos et al., 1994). LY294002 has also been shown to block PI3 kinase-dependent Akt phosphorylation and kinase activity. Although the reported IC₅₀ of LY294002 is about 500-fold higher than that of wortmannin, LY294002 is widely used in cell biology as a specific PI3K inhibitor because it is much more stable in solution than wortmannin. At concentrations at which LY294002 fully inhibits the ATP-binding site of PI3K, it has no inhibitory effect against a number of other ATP-requiring enzymes including PI4-kinase, EGF receptor tyrosine kinase, src-like kinases, MAP kinase, protein kinase A, protein kinase C, and ATPase.

B. MAPK and JNK Inhibitors

Among the key signaling pathways regulating mammalian cell growth and differentiation is the MKK/ERK pathway, comprised of MAP kinases, ERK1/2, and MAP kinase kinases, MKK1/2 (Lewis et al., 1998). JNK belongs to the family of MAPKs, of which ERK and p38 are well characterized homologous members. ERK1/2 and MKK1/2 are acutely stimulated by growth and differentiation factors in pathways mediated by receptor tyrosine kinases, heterotrimeric G protein-coupled receptors or cytokine receptors, primarily through p21Ras-coupled mechanisms. These enzymes are ubiquitous and are generally expressed at micromolar levels in mammalian cells (Huang and Ferrell, 1996), although some variation in expression between different tissues has been noted (Boulton and Cobb, 1991; Moriguchi et al., 1995). It has been demonstrated that different cell types utilize the MKK/ERK pathway to modulate responses as varied as cell proliferation, cell growth arrest and lineage-specific gene expression

Enhancement of MKK or ERK activity in response to cell stimulation involves phosphorylation at residues located within the activation lip of each kinase. In the case of MKK, phosphorylation at two serine residues (Ser²¹⁸/Ser²²² in human MKK1; Ser²²²/Ser²²⁶ in human MKK2) by upstream protein kinases, Raf-1, c-Mos or MEKK (M” K kinase kinase), leads to maximal enzyme activation. Subsequently, MKK1/2 activates ERK1/2 by phosphorylating regulatory threonine and tyrosine residues (Thr²⁰²/Tyr²⁰⁴ in hERK1; Thr¹⁸⁵/Tyr¹⁸⁷ in hERK2). Thus, MKKs fall within a relatively rare class of protein kinases with dual specificity toward Ser/Thr and Tyr residues on exogenous substrates.

Selective cell-permeable kinase inhibitors of the signal transduction provide a useful tool in the present invention. These include mitogen-activated protein kinase (MAPK)/extracellular signal-regulated kinase (ERK) kinase (MEK) inhibitors such as but are not limited to: PD98059, PD184352 and U0126 which are noncompetitive inhibitors of MEK1 and MEK2.

U0126 (1,4diamino-2,3-dicyano-1,4 bis[2-aminophenylthio]butadiene) was recently described as a novel inhibitor of MKK1 and MKK2 (Favata, 1998). The compound, identified in a screen for inhibitors of AP-1 transactivation in a cell-based reporter assay, inhibited phorbol 12-myristate 13-acetate (PMA)-induction of genes controlled by the 12-O-tetradecanoyl-phorbol 13-acetate (TPA) response element (TRE), at a concentration of 1-2 μM. U0126 inhibits both MKK1 and MKK2 at submicromolar concentrations in vitro, and appears to be more effective toward constitutively active MKK1/2 mutants than MKK activated by phosphorylation (Favata, 1998).

U0126 has properties in common with the widely used PD098059 inhibitor, sharing the ability to inhibit the MKK/ERK pathway in response to mitogenic stimulation. Unlike PD098059, U0126 exhibits similar potency for both MKK1 and MKK2, higher affinity for MKK binding and enhanced solubility in aqueous solution. In intact cells, U0126 blocks ERK activation at one-tenth the concentration of PD098059, and inhibits MKK activity without interfering with phosphorylation and activation of MKK. The available infornmation comparing inhibition of several protein kinases suggests selectivity for MKK1 and MKK2. PD098059 is a selective inhibitor of MKK1 and blocks MKK/ERK activation in intact cells. PD184352 inhibits cell cycle progression through inhibition of the ERK1/2 pathway.

Other MAPK and JNK inhibitors which may be employed in the present invention include but are not limited to: Ro092210, LLZ16402 and L783277 which are compounds isolated from microorganisms. Ro092210 and LLZ16402 are inhibitors of MEK1 and MEK2 that compete with ATP. L783277 has a similar structure to Ro092210 and LLZ16402. L783277 is reported to inhibit Jun-N-terminal kinase (JNK)/p38 MAPK pathways upstream of MAPK.

### IV. ANTICANCER THERAPY

In some embodiments, the present invention contemplates the use of a chemotherapeutic agent, such as taxol or doxorubicin, as a second agent in combination with deguelin or deguelin derivatives in treating or preventing lung cancer. In further embodiments, the second agent contemplated for use with deguelin or derivatives thereof may be a radiotherapeutic agent.

A. Taxol/Paclitaxel

Paclitaxel, also known as taxol is a diterpene alkaloid thus it possesses a taxane skeleton in its structure. Paclitaxel is extracted from the bark of the Pacific yew (Taxus brevifolia) as a natural compound having anti-cancer activity (Fuchs and Johnson, 1978). Paclitaxel works against cancer by interfering with mitosis. Paclitaxel is a taxoid drug, widely used as an effective treatment of primary and metastatic cancers.

Paclitaxel (Taxol) is widely used in the treatment of breast, ovarian, and other solid tumors. Randomized clinical trials have shown a survival advantage among patients with primary breast cancer who received paclitaxel in addition to anthracycline-containing adjuvant chemotherapy (Eifel et al., 2001). Furthermore, paclitaxel is effective for both metastatic breast cancer (Holmes et al.,-1991; Nabholtz et al., 1996; Bishop et al., 1999) and advanced ovarian cancer (McGuire et al., 1996; Piccart et al., 2000). The antitumor activity of paclitaxel is unique because it promotes microtubule assembly and stabilizes the microtubules, thus preventing mitosis (Huizing et al., 1995). Paclitaxel does this by reversibly and specifically binding to the B subunit of tubulin, forming microtubule polymers thereby stabilizing them against depolymerization and thus leading to growth arrest in the G2/M phase of the cell cycle (Gotaskie and Andreassi, 1994). This makes taxol unique in comparison to vincristine and vinblastine which cause microtubule disassembly (Gatzemeier et al., 1995). Additionally, recent evidence indicates that the microtubule system is essential to the release of various cytokines and modulation of cytokine release may play a major role in the drug's antitumor activity (Smith et al., 1995).

However, some patients are resistant to paclitaxel therapy, and the characteristics of patients who will benefit from the drug have not been well defined. Identification of molecular characteristics predictive of paclitaxel sensitivity or resistance could aid in selecting patients to receive this therapy. Thus, in particular embodiments, the present invention relates to paclitaxel sensitivity in a patient having cancer.

Previous reports have demonstrated that paclitaxel resistance is due to a variety of mechanisms such as up-regulation of anti-apoptotic Bcl-2 family members, such as Bcl-2 and Bcl-X_{L} (Tang et al., 1994); up-regulation of membrane transporters (e.g., mdr-1), resulting in an increased drug efflux (Huang et al., 1997); mutations in beta-tubulin resulting in abolishment of paclitaxel binding (Giannakakou et al., 1997); and up-regulation of ErbB2 (HER2) through inhibition of cyclin-dependent kinase-1 (Cdk1), resulting in delayed mitosis (Yu et al., 1998).

Due to the antimitotic activity of paclitaxel it is a useful cytotoxic drug in treating several classic refractory tumors. Paclitaxel has primarily been use to treat breast cancer and ovarian cancer. It may also be used in treating head and neck cancer, Kaposi's sarcoma and lung cancer, small cell and non-small cell lung cancer. It may also slow the course of melanoma. Response rates to taxol treatment varies among cancers. Advanced drug refractory ovarian cancer responds at a 19-36% rate, previously treated metastatic breast cancer at 27-62%, and various lung cancers at 21-37%. Taxol has also been shown to produce complete tumor remission in some cases (Guchelaar et al., 1994).

Paclitaxel is given intravenously since it irritates skin and mucous membranes on contact.

It is typically administered intravenously by a 3 to 24 hour infusion three times per week (Guchelaar et al., 1994).

B. Doxorubicin

Doxorubicin hydrochloride, 5,12-Naphthacenedione, (8s-cis)-10-[(3-amino-2,3,6-trideoxy-a-L-lyxo-hexopyranosyl)oxy]-7,8,9,10-tetrahydro-6,8,11-trihydroxy-8-(hydroxyacetyl)-1-methoxy-hydrochloride (hydroxydaunorubicin hydrochloride, Adriamycin) is used in a wide antineoplastic spectrum. It binds to DNA and inhibits nucleic acid synthesis and mitosis, and promotes chromosomal aberrations.

Administered alone, it is the drug of first choice for the treatment of thyroid adenoma and primary hepatocellular carcinoma. It is a component of first-choice in combination with other agents for the treatment of ovarian tumors, endometrial and breast tumors, bronchogenic oat-cell carcinoma, non-small cell lung carcinoma, gastric adenocarcinoma, retinoblastoma, neuroblastoma, mycosis fungoides, pancreatic carcinoma, prostatic carcinoma, bladder carcinoma, myeloma, diffuse histiocytic lymphoma, Wilms' tumor, Hodgkin's disease, adrenal tumors, osteogenic sarcoma soft tissue sarcoma, Ewing's sarcoma, rhabdomyosarcoma and acute lymphocytic leukemia. It is an alternative drug for the treatment of islet cell, cervical, testicular and adrenocortical cancers. It is also an immunosuppressant.

Since doxorubicin is poorly absorbed it is administered intravenously. The pharmacokinetics of this chemotherapeutic agent are multicompartnental. Distribution phases have half-lives of 12 minutes and 3.3 hr. The elimination half-life is about 30 hr. Forty to 50% is secreted into the bile. Most of the remainder is metabolized in the liver, partly to an active metabolite (doxorubicinol), but a few percent is excreted into the urine. In the presence of liver impairment, the dose should be reduced.

Appropriate doses are, for an adult, administered intravenously, are 60 to 75 mg/m² at 21-day intervals, or 25 to 30 mg/m² on each of 2 or 3 successive days repeated at 3- or 4-wk intervals, or 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by 50% if the serum bilirubin lies between 1.2 and 3 mg/dL and by 75% if above 3 mg/dL. The lifetime total dose should not exceed 550 mg/m² in patients with normal heart function and 400 mg/m² in persons having received mediastinal irradiation. Alternatively, 30 mg/m² on each of 3 consecutive days, repeated every 4 wk may be administered. Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the present invention.

C. Radiotherapy

Radiotherapy, also called radiation therapy, involves the use of ionizing radiation to treat cancers and other diseases. Ionizing radiation deposits energy that injures or destroys cells in the area being treated (the “target tissue”) by damaging their genetic material, and thereby inhibiting cell proliferation. Ionizing radiation induces the formation of hydroxyl radicals, placing the cells under oxidative stress. These radicals damage DNA, which causes cytotoxicity.

Radiotherapeutic agents that cause DNA damage are well known in the art and have been extensively used. Radiotherapeutic agents, through the production of oxygen-related free radicals and DNA damage, may lead to cell death or apoptosis. These agents may include, but are not limited to, γ-rays, X-rays, and/or the directed delivery of radioisotopes to tumor cells (known as internal radiotherapy). Internal radiotherapy may further include but is not limited to, brachytherapy, interstitial irradiation, and intracavitary irradiation. Other radiotherapeutic agents that are DNA damaging factors include microwaves and UV-irradiation. These factors effect a broad range of damage on DNA, on the precursors of DNA, on the replication and repair of DNA, and on the assembly and maintenance of chromosomes.

Other approaches to radiation therapy are also contemplated in the present invention. Such techniques may comprise intraoperative irradiation, in which a large dose of external radiation is directed at the tumor and surrounding tissue during surgery; and particle beam radiation therapy which involves the use of fast-moving subatomic particles to treat localized cancers. Radiotherapy may further involve the use of radiosensitizers and/or radioprotectors to increase the effectiveness of radiation therapy. Radiolabeled antibodies may also be used to deliver doses of radiation directly to the cancer site, this is known as radioimmunotherapy.

Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### V. ADJUNCT CANCER THERAPY

In the context of the present invention, it is contemplated that deguelin or derivatives thereof may be used in combination with a second agent. It is further contemplated that the second agent may be a PI3K, MAPK, or JNK inhibitor or an anticancer therapy such as taxol, doxorubicin or radiotherapy. It may also prove effective to combine deguelin and a second agent with an adjunct agent such as chemotherapy, gene therapy, hormonal therapy or immunotherapy that targets cancer/tumor cells.

To kill cells, inhibit cell growth, inhibit metastasis, inhibit angiogenesis or otherwise reverse or reduce the malignant phenotype of cancer cells, using the methods and compositions of the present invention, one would generally contact a cell with deguelin or derivatives thereof in combination with a second agent such as a PI3K, MAPK, or JNK inhibitor; or an anticancer therapy such as taxol, doxorubicin or radiotherapy. All of these compositions would be provided in a combined amount effective to kill or inhibit proliferation of the cell. This process may involve contacting the cells with deguelin or derivatives thereof in combination with a second agent or factor(s) at the same time. This may be achieved by contacting the cell with a single composition or pharmacological formulation that includes both agents, or by contacting the cell with two distinct compositions or formulations, at the same time, wherein one composition includes the deguelin or derivates thereof and the other includes the second agent.

Alternatively, treatment with deguelin or a deguelin derivative may precede or follow the second agent treatment by intervals ranging from minutes to weeks. In embodiments where the second agent is applied separately to the cell, one would generally ensure that a significant period of time did not expire between the time of each delivery, such that the agent would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either deguelin, or derivatives thereof in combination with a second agent such as a PI3K, MAPK, or JNK inhibitor; or anticancer therapy such as taxol, doxorubicin or radiotherapy will be desired. Various combinations may be employed, where deguelin or derivatives thereof is “A” and the second agent is “B”, as exemplified below:

| | | | | | | | |
|---|---|---|---|---|---|---|---|
| A/B/A | B/A/B | B/B/A | A/A/B | B/A/A | A/B/B | B/B/B/A | B/B/A/B |

| | | | | | | |
|---|---|---|---|---|---|---|
| A/A/B/B | A/B/A/B | A/B/B/A | B/B/A/A | B/A/B/A | B/A/A/B | B/B/B/A |
| A/A/A/B | B/A/A/A | A/B/A/A | A/A/B/A | A/B/B/B | B/A/B/B | B/B/A/B |

Other combinations are contemplated. Again, to achieve cell killing, both agents are delivered to a cell in a combined amount effective to kill the cell.

As stated above, a further combination with adjunct therapies is envision. Adjunct agents or factors suitable for use in combination with the present invention include any chemical compound or treatment method with anticancer activity. These compounds or methods include alkylating agents, topoisomerase I inhibitors, topoisomerase II inhibitors, antitumor antibiotics, RNA/DNA antimetabolites, DNA antimetabolites, antimitotic agents, nitrosureas, as well as antibodies and corticosteroid hormones.

a. Chemotherapy

All adjunct therapy contemplated in the present invention is chemotherapy. Adjunct chemotherapies may include, for example, cisplatin (CDDP), carboplatin, procarbazine, mechloretharnine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

b. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

c. Genes

In yet another embodiment, the secondary treatment is a secondary gene therapy in which a second therapeutic polynucleotide is administered before, after, or at the same time a first therapeutic polynucleotide encoding all of part of an MDA-7 polypeptide. Delivery of a vector encoding either a full length or truncated MDA-7 in conjuction with a second vector encoding one of the following gene products will have a combined anti-hyperproliferative effect on target tissues. Alternatively, a single vector encoding both genes may be used. A variety of proteins are encompassed within the invention, some of which are described below.

i. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

ii. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16mK4 protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, mda-7, APC, DCC, NF-1, NF-2, WT-1, MEN-I, MEN-II, zacl, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf erb, fms, trk, ret, gsp, hst, abl, E1A, p300, genes involved in angiogenesis (e.g., VEGF, FGF, thrombospondin, BAI-1, GDAIF, or their receptors) and MCC.

iii. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., BCl_{XL}, BCl_{w}, Bclₛ, Mcl-1, Al, Bfl-1) or counteract Bcl-2 function and promote cell death (e.g., Bax, Bak, Bik, Bim, Bid, Bad, Harakiri).

d. Surgery

Approximately 60% of persons with cancer will undergo surgery of some type, which includes preventative, diagnostic or staging, curative and palliative surgery. Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

e. Hormonal Therapy

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

f. Other agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adehesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1 beta, MCP-1, RANTES, and other chemokines. It is further contemplated that the upregulation of cell surface receptors or their ligands such as Fas/Fas ligand, DR4 or DR5/TRAIL would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

B. Formulations and Routes for Administration

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention in an effective amount may be dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier”, includes any and all solvents, dispersion media, coatings, antibacterial and antifungal agents, isotonic and absorption delaying agents and the like. The use of such media and agents for. pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The composition(s) of the present invention may be delivered orally, nasally, intramuscularly, intraperitoneally, In some embodiments, local or regional delivery of deguelin or derivatives thereof in combination with a second agent, to a patient with cancer or pre-cancer conditions will be a very efficient method of delivery to counteract the clinical disease. Similarly, chemo- or radiotherapy may be directed to a particular, affected region of the subject's body. Regional chemotherapy typically involves targeting anticancer agents to the region of the body where the cancer cells or tumor are located. Other examples of delivery of the compounds of the present invention that may be employed include intra-arterial, intracavity, intravesical, intrathecal, intrapleural, and intraperitoneal routes.

Intra-arterial administration is achieved using a catheter that is inserted into an artery to an organ or to an extremity. Typically, a pump is attached to the catheter. Intracavity administration describes when chemotherapeutic drugs are introduced directly into a body cavity such as intravesical (into the bladder), peritoneal (abdominal) cavity, or pleural (chest) cavity. Agents can be given directly via catheter. Intravesical chemotherapy involves a urinary catheter to provide drugs to the bladder, and is thus useful for the treatment of bladder cancer. Intrapleural administration is accomplished using large and small chest catheters, while a Tenkhoff catheter (a catheter specially designed for removing or adding large amounts of fluid from or into the peritoneum) or a catheter with an implanted port is used for intraperitoneal chemotherapy. Abdomen cancer may be treated this way. Because most drugs do not penetrate the blood/brain barrier, intrathecal chemotherapy is used to reach cancer cells in the central nervous system. To do this, drugs are administered directly into the cerebrospinal fluid. This method is useful to treat leukemia or cancers that have spread to the spinal cord or brain.

Alternatively, systemic delivery of the chemotherapeutic drugs may be appropriate in certain circumstances, for example, where extensive metastasis has occurred. Intravenous therapy can be implemented in a number of ways, such as by peripheral access or through a vascular access device (VAD). A VAD is a device that includes a catheter, which is placed into a large vein in the arm, chest, or neck. It can be used to administer several drugs simultaneously, for long-term treatment, for continuous infusion, and for drugs that are vesicants, which may produce serious injury to skin or muscle. Various types of vascular access devices are available.

The active compositions of the present invention may include classic pharmaceutical preparations. Administration of these compositions according to the present invention will be via any common route so long as the target tissue is available via that route. This includes but is not limited to, oral, nasal, or buccal routes. Alternatively, administration may be by orthotopic, intradermal, subcutaneous, intramuscular, intraperitoneal or intravenous injection. Such compositions would normally be administered as pharmaceutically acceptable compositions, described supra. The drugs and agents also may be administered parenterally or intraperitoneally. The term “parenteral” is generally used to refer to drugs given intravenously, intramuscularly, or subcutaneously.

Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The therapeutic compositions of the present invention may be administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH, exact concentration of the various components, and the pharmaceutical composition are adjusted according to well known parameters. Suitable excipients for formulation with deguelin or derivatives thereof in combination a second agent include croscannellose sodium, hydroxypropyl methylcellulose, iron oxides synthetic), magnesium stearate, microcrystalline cellulose, polyethylene glycol 400, polysorbate 80, povidone, silicon dioxide, titanium dioxide, and water (purified).

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

An effective amount of the therapeutic agent(s) is determined based on the intended goal, for example (i) inhibition of tumor cell proliferation or (ii) elimination of tumor cells. The term “unit dose” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

C. Therapeutically Effective Amounts of Deguelin and Derivatives Thereof

Treatment or prevention of a lung cancer with a therapeutically effective amount of a deguelin or derivatives thereof in combination with a second agent such as a PI3K, MAPK, or JNK inhibitor, or an anticaner therapy such as taxol, doxorubicin or radiotherapy varies depending upon the host treated and the particular mode of administration. In one embodiment of the invention the dose range of a deguelin or derivatives thereof in combination with a second agent used will be about 0.5 mg/kg body weight to about 500 mg/kg body weight. The term “body weight” is applicable when an animal is being treated. When isolated cells are being treated, “body weight” as used herein should read to mean “total cell weight”. The term “total weight may be used to apply to both isolated cell and animal treatment. All concentrations and treatment levels are expressed as “body weight” or simply “kg” in this application are also considered to cover the analogous “total cell weight” and “total weight” concentrations. However, those of skill will recognize the utility of a variety of dosage range, for example, 1 mg/kg body weight to 450 mg/kg body weight, 2 mg/kg body weight to 400 mg/kg body weighty, 3 mg/kg body weight to 350 mg/kg body weighty, 4 mg/kg body weight to 300 mg/kg body weight, 5 mg/kg body weight to 250 mg/kg body weighty, 6 mg/kg body weight to 200 mg/kg body weight, 7 mg/kg body weight to 150 mg/kg body weighty, 8 mg/kg body weight to 100 mg/kg body weight, or 9 mg/kg body weight to 50 mg/kg body weight. Further, those of skill will recognize that a variety of different dosage levels will be of use, for example, 1 mg/kg, 2 mg/kg, 3 mg/kg, 4 mg/kg, 5 mg/kg, 7.5 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 120 mg/kg, 140 mg/kg, 150 mg/kg, 160 mg/kg, 180 mg/kg, 200 mg/kg, 225 mg/kg, 250 mg/kg, 275 mg/kg, 300 mg/kg, 325 mg/kg, 350 mg/kg, 375 mg/kg, 400 mg/kg, 450 mg/kg, 500 mg/kg, 550 mg/kg, 600 mg/kg, 700 mg/kg, 750 mg/kg, 800 mg/kg, 900 mg/kg, 1000 mg/kg, 1250 mg/kg, 1500 mg/kg, 1750 mg/kg, 2000 mg/kg, 2500 mg/kg, and/or 3000 mg/kg. Of course, all of these dosages are exemplary, and any dosage in-between these points is also expected to be of use in the invention. Any of the above dosage ranges or dosage levels may be employed for deguelin or derivatives thereof in combination with second agent.

“Therapeutically effective amounts” are those amounts effective to produce beneficial results, particularly with respect to cancer treatment, in the recipient animal or patient. Such amounts may be initially determined by reviewing the published literature, by conducting in vitro tests or by conducting metabolic studies in healthy experimental animals. Before use in a clinical setting, it may be beneficial to conduct confirmatory studies in an animal model, preferably a widely accepted animal model of the particular disease to be treated. Preferred animal models for use in certain embodiments are rodent models, which are preferred because they are economical to use and, particularly, because the results gained are widely accepted as predictive of clinical value.

As is well known in the art, a specific dose level of active compounds such as deguelin or derivatives thereof in combination with a second agent, for any particular patient depends upon a variety of factors including the activity of the specific compound employed, the age, body weight, general health, sex, diet, time of administration, route of administration, rate of excretion, drug combination, and the severity of the particular disease undergoing therapy. The person responsible for administration will determine the appropriate dose for the individual subject. Moreover, for human administration, preparations should meet sterility, pyrogenicity, general safety and purity standards as required by FDA Office of Biologics standards.

In some embodiments, deguelin or derivatives thereof in combination with a second agent will be administered. As long as the dose of the second agent does not exceed previously quoted toxicity levels, the effective amounts of the second agents may simply be defined as those amounts effective to reduce the cancer growth when administered to an animal in combination with the deguelin or derivatives thereof This is easily determined by monitoring the animal or patient and measuring those physical and biochemical parameters of health and disease that are indicative of the success of a given treatment. Such methods are routine in animal testing and clinical practice.

In some embodiments of the present invention chemotherapy may be administered, as is typical, in regular cycles. A cycle may involve one dose, after which several days or weeks without treatment ensues for normal tissues to recover from the drug's side effects. Doses may be given several days in a row, or every other day for several days, followed by a period of rest. If more than one drug is used, the treatment plan will specify how often and exactly when each drug should be given. The number of cycles a person receives may be determined before treatment starts (based on the type and stage of cancer) or may be flexible, in order to take into account how quickly the tumor is shrinking. Certain serious side effects may also require doctors to adjust chemotherapy plans to allow the patient time to recover.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similarresult without departing from the spirit and scope of the invention.

### Example 1

### Materials and Methods

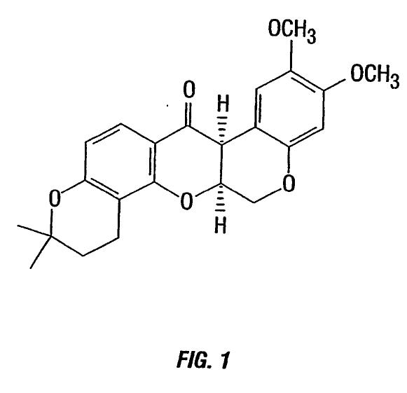

Preparation of Deguelin. Deguelin (FIG. 1) was synthesized from the natural product rotenone (Sigma-Aldrich, Milwaukee, Wis.) in four steps to provide material in >98% pure, as previously described (Anzenveno, 1979).

Cells and Cell Cultures. A lung carcinogenesis model that includes normal, premalignant, and malignant HBE cells was used in this study. Normal HBE (NHBE) cells were purchased from Clontech (Palo Alto, Calif.). For the purpose of this study, premalignant cell lines were defined as immortalized nontumorigenic HBE cells (1799 cells) or inunortalized nontumorigenic HBE cells exposed to carcinogen (1198 cells), and malignant cell lines were defined as immortalized tumorigenic HBE cells (1170 cells). The premalignant and malignant cell lines were derived from a single-cell subclone of the BEAS-2B cell line, which is an HBE cell immortalized with a hybrid adenovirus/simian Virus 40 (Reddel et al., 1988). To develop the immortalized and tumorigenic HBE cell lines, BEAS-2B cells were inserted into rat tracheas that had been denuded of bronchial epithelium; beeswax pellets containing either cigarette smoke condensate (CSC) or no treatment were also inserted into the rat tracheas. The tracheas were placed subcutaneously in nude mice. Tumors developed 6 months later. Cell lines that exhibited various levels of tumorigenicity in nude mice were derived from the tumors. 1799 is a nontumorigenic cell line derived from BEAS-2B cells exposed to a beeswax pellet alone. Cell lines derived from BEAS-2B cells exposed to beeswax pellets containing CSC include the 1198 cell line, which is nontumorigenic, and the 1170-1 cell line, which is tumorigenic. Tumorigenic 1 170-1 cells exhibit an adenocarcinoma appearance. The 1799, 1198, and 1170-1 were obtained from Dr. Andres Klein-zanto, Fox Chase Cancer Center, Philadelphia, Pa. (Klein-Szanto et al., 1992). The characteristics of these cell lines have been described in detail (Kim et. al., 1995). Squamous HBE cells were induced by growing HBE cells to confluence on 10-cm tissue culture plates coated with a thin matrix of fibronectin (Upstate Biotechnology, Inc., Lake Placid, N.Y.) and collagen (Celtrix Laboratories, Inc., Palo Alto, Calif.) as previously described (Lee et al., 1996). The NH1BE cells, 1799 cells, and squamous HBE cells were grown in keratinocyte serum-free medium (KSFM; Life Technologies, Inc., Gaithersburg, Md.) containing 2 μg/ml of epidermal growth factor (EGF) and bovine pitutary extract (Reddel et al., 1988), whereas 3% serum is required for the growth of 1198 and 1170-1 cells (20). Cells were grown on tissue culture plasticware (Falcon; Bector, Dickinson, Bedford, Mass.) at 37° C. in a humidified atmosphere with 5% C0₂. For the analyses of growth inhibition, cell cycle, and induction of apoptosis by deguelin, NHBE cells, HBE cell lines, and squamous HBE cells were cultured in KSFM (Life Technologies) containing 2 μg/ml of EGF and bovine pitutary extract.

Cell Treatment with Deguelin and Determination of Growth Inhibition. To measure the effects of deguelin on cell proliferation, NHBE, 1799 cells, 1198 cells, and 1170 cells were transferred onto 96-well plates at densities ranging from 2×10³ to 4×10³ cells/well. After 1 day, the cells were changed to the fresh medium containing various concentrations of deguelin dissolved in DMSO (final concentration, 0.1%). Control cultures received 0.1% dimethyl sulfoxide (DMSO) as did the deguelin-treated cultures. To determine whether deguelin-induced antiproliferative effects on premalignant HBE cells was mediated through the inhibition of PI3K/Akt pathway, 1799 cells and HBE cells were transferred onto 96-well plates, and infected with Ad5CMV (5×10³ particles/cell), an empty virus, or Ad5CMV-MyrAkt-HA (1×10³ or 5×10³ particles/cell), an adenoviral vector expressing constitutively active Akt (MyrAkt), in KSFM. After 1 day of infection, cells were treated with 10⁻⁷ M or 10⁻⁶ M of deguelin, or 0.1% DMSO as a control, and then incubated for 2 days. The viability of treated cells was measured by 3-(4,5-dimethylthiazol-2-yl)-2,5-diphenyltetrazolium bromide (MTT) assay as previously described (Lee et al., 2002). Six replicate wells were used for each analysis. The drug concentration required to cause 50% cell growth inhibition (IC₅₀) was determined by interpolation from dose-response curves.

Cell Cycle Analysis. Cells were plated on 10-cm dishes 1 day before treatment. After treatment with deguelin for 3 days, floating and adherent cells were harvested by trypsinization, fixed with 1% paraformaldehyde and 70% ethanol, stained with propidium iodide PI), and subjected to flow cytomnetric analysis to determine the percentages of cells in specific phases of the cell cycle (G₁, S, and G₂/M) as described previously (Sun et al., 1997).

Apoptosis assay. Normal, premalignant, and malignant HBE cells were exposed to v various doses of deguelin for 3 days. Morphologic characteristics of the cells were observed with a light microscope at ×200. Both adherent and floating cells were combined for the assessment of apoptosis using the APO-BRDU staining kit (Phoenix Flow Systems, San Diego, Calif.). Briefly, floating and attached cells dispersed with trypsin-EDTA were pelleted, washed, and fixed by 1% paraformaldehyde followed by 70% ethanol. The fixed cells were washed and incubated with DNA-labeling solution containing terminal deoxynucleotidyl transferase (TdT) reaction buffer, TdT enzyme, and bromodeoxyuridine triphosphate (Br-dUTP). Cells were rinsed, resuspended with fluorescein-PRB-I antibody solution, and analyzed by flow cytometry in the presence of PI/RNase solution. All analyses were performed based on 3000 to 10,000 events using a FACScan flow cytometer (Becton Dickinson, San Jose, Calif.) equipped with a 488-nm argon ion laser and CellQuest software. A dual display of DNA area (linear red fluorescence) and Br-dUTP incorporation (FITC-PRB-1) was used to determine the percentage of apoptotic cells.

The percentage of dead cells was determined by fluorescent-activated cell sorting (FACS) analysis of PI-stained nuclei. Apoptosis was also determined by the detection of nucleosomal DNA fragmentation, which was measured using the TACS apoptotic DNA Jaddering kit (Trevigen, Inc., Gaithersburg, Md.) according to the manufacturer's protocol. To determine whether deguelin-induced apoptosis was mediated through the inhibition of the PI3K/Akt pathway, 2×10⁵ 1799 cells or squamous HBE cells were seeded onto 6-well plates. After 1 day, cells were infected with Ad5CMV (5×10³ particles/cell) or Ad5CMV-MyrAkt-HA (1×10³ or 5×10³ particleslcell) in KSFM, followed by the treatment with either 10⁻⁷M deguelin or 0.1% DMSO as a control, and then incubated for 2 days. Apoptosis was analyzed using the APO-BRDU staining kit (Phoenix Flow Systems) as described above.

Immunoblotting. Whole cell lysates were prepared in lysis buffer (50 mM N-[2-hydroxymethyl]-piperazine-N′-[2-ethanesulfonic acid] [HEPES; pH 7.5], 150 mM NaCl, 1.5 mM MgCl₂, ImM EDTA, 0.2 mM EGTA, 1% NP40, 10% glycerol, 1 mM dithiothreitol (DTT), 1 mM phenylmethylsulfonyl fluoride, 20 mM sodium fluoride, 5 mM sodium orthovanadate, 10 μg/ml aprotinin, 10 μg/ml leupeptin, 2 μg/ml pepstatin, and 1 mM benzamidine) as described previously (Lee et al., 2002). Equivalent protein concentrations were resolved in sodium dodecyl sulfate-polyacrylamide gels and transferred to a nitrocellulose membrane. After the blocking of transblotted membrane in Tris-buffered saline (TBS) containing 0.05% Tween 20 (TBST) and 5% low fat milk, themembrane was incubated with primary antibody at the appropriate dilution in TBS-5% low-fat milk at 4° C. for 16 h and washed with TBST. The immunoblots were visualized using the ECL kit (Amersham, Inc., Arlington Heights, III) according to the manufacturer's directions. Rabbit polyclonal antibodies against human pAkt (Ser473), Akt, and pGSK-3β (Ser9), and mouse monoclonal antibody against human anti-pMAPK (Thr202/Tyr204) were purchased from Cell Signalinng Technology (Beverly, Mass.). Rabbit polyclonal anti-glycogen synthase (GSK)-3 α/β (BD Transduction Laboratories, Lexington, Ky.), rabbit polyclonal anti-Bax and anti-caspase-3 antibodies (Pharmingen, San Diego, Calif.), rabbit polyclonal anti-Bcl2 and rabbit polyclonal anti-poly (ADP-ribose) polymerase O?ARP) antibody (VIC 5) (Roche Molecular Biochemicals, Indianapolis, Ind.), goat antibodies against Erk-1, Erk-2, and β-Actin (Santa Cruz Biotechnology, Inc., Santa Cruz, Calif.) were used for western blot analysis.

PI3K Assay. 1799 cells cultured in KSFM containing 10⁻⁷ M deguelin for different time periods were lysed in lysis buffer. PI3K was immunoprecipitated from 500 μg of cellular protein using pan-anti-p85 antibody (Upstate Biotechnology, Waltham, Mass.), which coprecipitates the p110 catalytic subunit of PI3K, and subsequent lipid kinase assay was performed as described previously (Sibilia et al., 2000). Briefly, the mixture was incubated with gentle rocking at 4° C. for 12 h, 10 mg of protein A-sepharose (Amersham Pharmacia Biotech) were added, and the incubation was continued for another 2 h. The immunoprecipitates were washed, in tandem, three times with lysis buffer, twice with 0.1 M Tris/HCl, pH 7.5, containing 0.5 M LiCl, and 10 μM sodium vanadate, and twice with 10 mM Tris/HCl, pH 7.5, containing 100 MM NaCl, 10 μM sodium vanadate, and 1 mM EDTA. Adequate amounts of the washed antibody conjugates, in 10 μl, were added to 80 μl of 30 mM Hepes, pH 7.5, containing 125 μM ATP, 10 μCi of [α-³²P] ATP, and 6.25 mM MgCl₂, and the reaction was initiated by adding 20 μg of bovine brain extract (Type 1; Sigma) suspended in 10 μl of 30 mM Hepes, pH 7.5. After 10 min at 37° C., the reaction was terminated by adding 5 μl of 1 M EDTA and 25 μl of 5 M HCl followed by 160 μl of chloroform:methanol (1:1; v/v). Samples were centrifuged at 6,000×g for 5 min, and the lower organic phase was removed, applied to 1% oxalic acid-treated TLC plates, and then developed with n-propanol:2 M acetic acid (65:35) overnight. After drying, spots were located by autoradiography and compared with standards. The autoradiograms were scanned by a Photodyne image system and quantified using the NIH Image program (version 1.59).

ERK 1/2 Kinase assay. ERK1/2 activity was determined by analyzing MAPK-induced phosphorylation of myelin basic protein (MBP) as previously described (Lee et al., 2002). Briefly, 1799 cells cultured in KSFM containing 10⁻⁷ M deguelin for different time periods were lysed in lysis buffer, and ERK-1 and -2 were immunoprecipitated from 100 μg of cell extracts with antibodies (1 μg) that recognize ERK-1 and -2 (Santa Cruz Biotechnology) by rotation at 4° C. for overnight. The total volume was adjusted to 0.5 ml with lysis buffer. Protein A sepharose beads (20 μl) (Amersham Pharmacia Biotech) were added and incubated at 4° C. for 2 hour. The beads were washed three times with lysis buffer and once with kinase buffer (20 MM Hepes [pH 7.5], 20 mM β-glycerol phosphate, 10 mM PNPP, 10 mM MgCl₂, 1 mM dithiothreitol, 50 mM sodium vanadate). Kinase assays were performed by incubating the beads with 30 μl kinase buffer to which 20 mM cold ATP, 5 μCi [γ³²P] ATP (2000 cpm/pmol), and 2 μg MBP (Cell Signaling Technology) were added. The kinase reaction was performed at 30° C. for 20 min. The samples were suspended in Laemmli buffer, boiled for 5 min, and the samples were analyzed by SDS-PAGE. The gel was dried and autoradiographed.

Generation of Ad5CMV-HA-Myr-Ak. An adenoviral vector expressing a full-length human Akt with the Src myristoylation signal fused in-frame to the c-Akt coding sequence with HA (MyrAkt-HA) (Franke et al., 1995) under the control of cytomegalovirns (CMV) promoter (AdSCMV-MyrAkt-HA) was constructed using the pAd-shuttle vector system, as previously described (Ji et al., 2002). The presence of MyrAkt-HA was confirmed by dideoxy-DNA sequencing and western blot analysis on Akt and HA. The function of Ad5CMV-MyrAkt-HA was examined by a western blot analysis on pGSK-3β (Ser9). Viral titers were determined by plaque assays and spectrophotometric analysis. The vectors for adenovirus construction were kindly provided by Dr. Jack A. Roth (The University of Texas M. D. Anderson Cancer Center, Houston, Tex.).

Northern Analysis. NHBE cells and squamous-HBE cells were lysed in 4.0 M guanidinium isothiocyanate and total cellular RNA was extracted as described previously (Lee et al., 1996). RNA was subjected to electrophoresis (20 μg per lane) on a 1% agarose gel containing 2% formaldehyde, transferred to a nylon membrane (Zeta-Probe, Bio-Rad), and hybridized to an [α-³²P]dCTP-labeled transglutaminase (TG) or involucrine (Inv) cDNA. Equal

loading of each RNA sample was examined by observing the intensity of 18s and 28s.

### Example 2

### Deguelin Inhibits Cell Growth Proliferation in HBE Cells

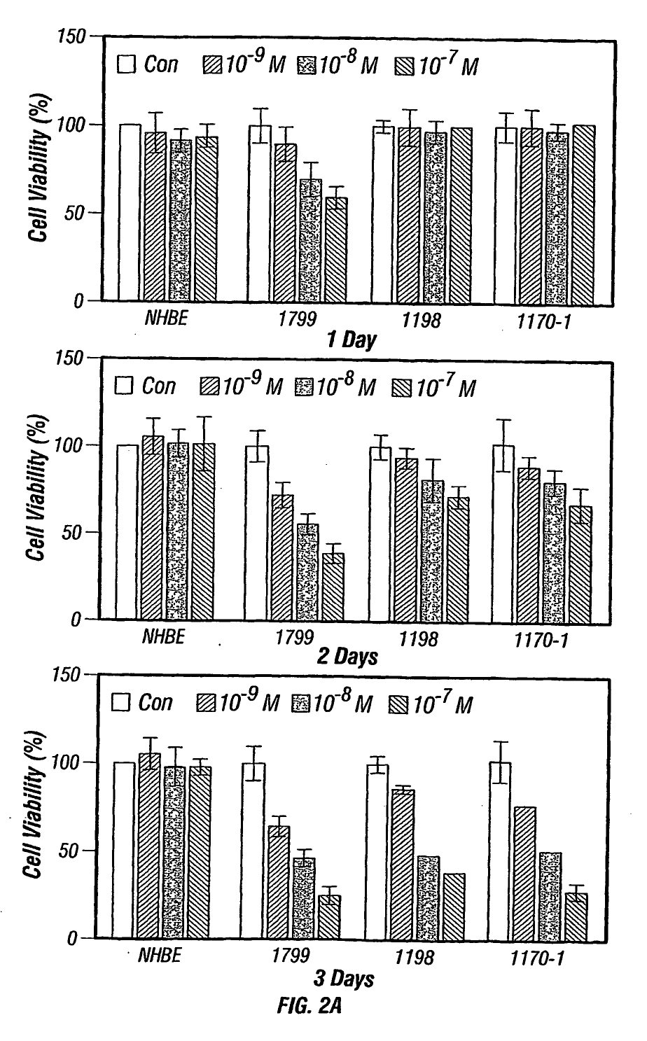

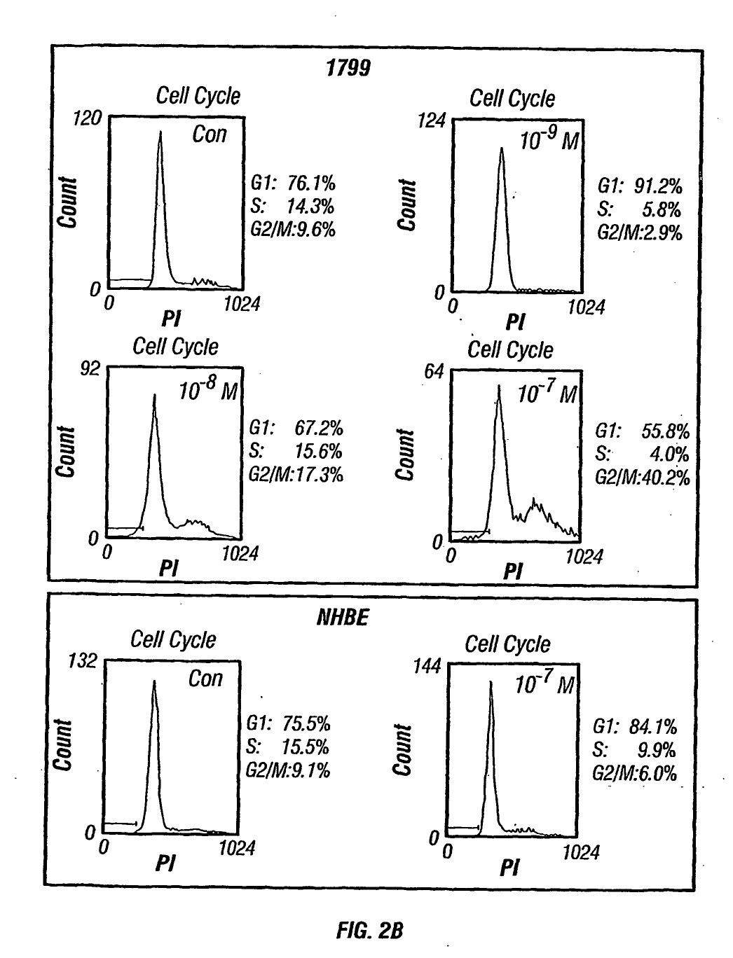

Differential Responses of Normal, Premalignant, and Malignant HBE Cells to Deguelini. To investigate the potential of deguelin as a lung cancer chemopreventive agent, the effects of deguelin on the growth of NHBE, two premalignant HBE cell lines, and one malignant HBE cell line, which together constitute an in vitro lung carcinogenesis model were examined. In the MTT assay after 3 days of treatment, deguelin inhibited the growth of premalignant and malign ant HBE cell lines at a concentration range of 10⁻⁹ M to 10⁻⁷ M (IC₅₀<10⁻⁹ M) in a dose- and time-dependent manner (FIG. 2A). The premalignant 1799 cells were the most sensitive to deguelin; the viable number of 1799 cells was reduced by treatment of deguelin for 1 day at concentration as low as 10⁻⁹ M. In contrast, deguelin had a minimal effect on NHBE viability, suggesting that deguelin acts specifically on neoplastically transformed HBE cells. Flow cytometry was performed to further characterize the effects of deguelin on cell proliferation. Cell cycle arrest in the G2/M phase was observed in 1799 cells treated with deguelin for 3 days at a range of concentration: 17.3% and 40.2% of the 1799 cells treated with 10⁻⁸ M to 10⁻⁷ M deguelin, respectively, were accumulated at the G2/M phase compared with the 9.6% of 1799 cells treated with DMSO (FIG. 2B). Analysis of 1198 and 1170 cells treated with deguelin in a same condition showed similar pattern in cell cycle distribution (data not shown), whereas deguelin did not induce detectable change in NHBE cell cycle. Thus, the results of the present invention demonstrate that deguelin significantly inhibits the growth of premalignant HBE cells as well as malignant HBE cells with minimal cytotoxicity to normal HBE cells and that premalignant 1799 cells were the most sensitive to deguelin-induced antiproliferative effects of deguelin, indicating the potential of deguelin as a chemopreventive agent against lung cancer. The mechanism through which deguelin inhibits cell growth was investigated, and it was found that deguelin treatment led to G2/M cell cycle arrest and rapid apoptosis in premalignant and malignant HBE cells in dose- and time-dependent manner, while it had little effects on normal HBE cells treated in a similar fashion.

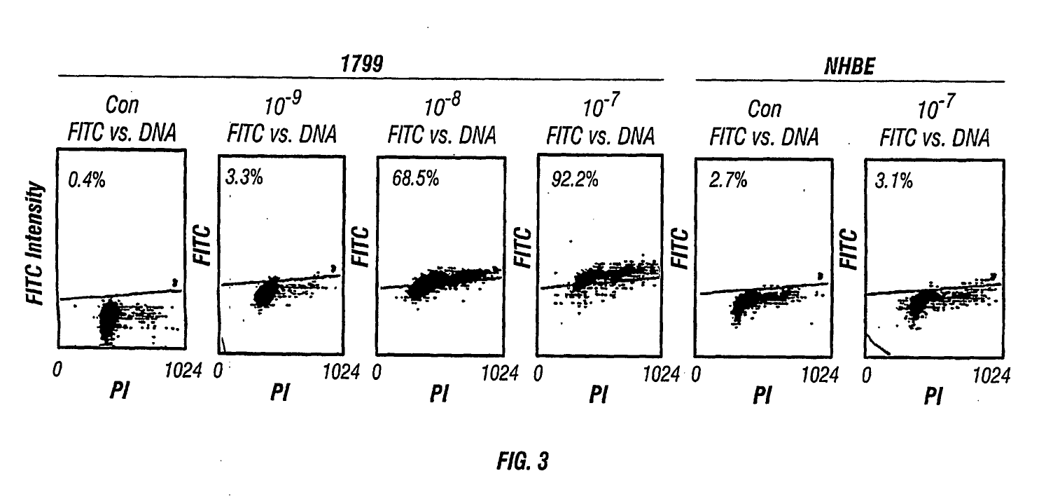

Induction of Apoptosis by Deguelin in vitro. The mechanism by which deguelin inhibits the growth of premalignant and malignant HBE cells was investigated. After treating 1799 cells with deguelin for 3 days, typical morphological changes of apoptosis was observed, including membrane blebbing, increased refractoriness, and chromatin condensation (data not shown). Flow cytometry, following the terminal deoxynucleotidyl transferase (TdT)-mediated dUTP-biotin nick end labeling (TUNEL), showed that deguelin has a potent apoptotic activity of deguelin in 1799 cells (FIG. 3). Exposure to more than 10⁻⁹ M deguelin for 3 days induced significant levels of apoptosis in 1799 cells; 10⁻⁸ M deguelin induced apoptosis in 68.5% of 1799 cells, and 10⁻⁷ M deguelin induced apoptosis in more than 90% of cells. Programmed cell death produces a characteristic pattern of DNA fragmentation resulting from cleavage of nuclear DNA; thus, DNA fragmentation was also assessed. Three days of treatment with deguelin induced the generation of nucleosomal-sized ladders of DNA fragments in the 1799 cells in a dose-dependent manner. DNA fragmentation was also observed in 1799 cells treated with 10⁻⁷ M deguelin for 1 day. 1198 and 1170 cells treated with same concentrations of deguelin for 3 days showed similar patterns in TUNEL and DNA fragmentation analyses, whereas treatment of deguelin for 1 day did not induce apoptotic events in 1198 and 1170 cells (data not shown), confirming the sensitivity of 1799 cells to deguelin. Consistent with the results from MTT assay, NHBE cells treated with deguelin showed neither TUNEL-positive cell population nor DNA fragmentation by deguelin treatment. Western blot analysis was also performed to determine whether deguelin induces an activation of the caspase-3, a key executioner of apoptosis, and cleavage of PARP, a substrate of caspase-3 proteolysis. A significant decrease in the 32-kDa caspase-3 proenzyne accompanied by a concomitant increase in the induction of the 89-kDa fragment of PARP cleaved from the 113-kDa form of PARP were shown in 1799 cells treated with more than 10⁻⁸ M deguelin for 3 days. The regulation of Bcl protein family by deguelin in 1799 cells was further analyzed. Deguelin induced a dose-dependent increase in the level of Bax, in association with the mild decrease in the Bcl-2 expression in these cells, whereas the Bcl-xL level was not affected. Similar effects of deguelin on the regulation of these proteins in 1198 and 1170 cells treated with deguelin in same condition were observed (data not shown).

Thus, the present invention demonstrates that deguelin induces the increase in the expression of Bax (Miyashita and Reed, 1995) and decrease in Bcl-2 in premalignant and malignant HBE cells, suggesting that changes in the ratio of Bax:Bcl-2 contribute to the apoptotic activity of deguelin in these cells. However, the modulation of Bcl family by deguelin was also observed in malignant HBE cells treated under the same condition, which suggested the presence of another mechanism that is responsible for the sensitivity of premalignant 1BE cells to deguelin.

### Example 3

### Effect of Deguelin on AKT Expression and Activity

PI3K/Akt Pathway is Constitutive Active in Premalignant HBE Cells. To explore the mechanism responsible for the induction of apoptosis by deguelin in 1799 cells, PI3K and MAPK, which have a major role in regulating cell proliferation and apoptosis (Robinson et al., 1997; Rodriguez-Viciana et al., 1997), were investigated to determine their involvment in deguelin-mediated apoptosis in 1799 cells. The level of phospho-Akt (pAkt) on Ser473 and phospho-P44/42 MAPK (pP44/42 MAPK) on Thr202/Tyr204 were examined in normal, premalignant, and malignant HBE cells that were incubated in serum-free KSFM for 1 day to remove exogenous activators of PI3K/Akt and MAPK. The level of pAkt was higher in premalignant and malignant HBE cells than in NHBE cells, whereas pP44/42 MAPK (Thr202/Tyr204) level was same in these cells. The1799 cells displayed the highest level of pAkt (S473) in growth factor withdrawal condition. To ensure that NHBE cells that did not exhibit S473 phosphorylation were capable of phosphorylating S473 upon stimulation, IGF-I was added, and S473 phosphorylation was measured. IGF-I increased S473 phosphorylation of NHBE cells irrespective of endogenous levels, indicating that the IGF-IR signaling pathway that leads to Akt activation is intact in NHBE cells. The fact that S473 phosphorylation was maintained in premalignant and malignant HBE cell lines under growth factor withdrawal indicated that Akt was constitutively active in these cells. The highest level of pAkt in 1799 cells suggested that the PI3K/Akt pathway plays an important role in cell survival in this cell line.

Inhibition of PI3K/Akt Activity by Deguelin in Premalignant HBE Cells. In determining the mechanism that mediates the effects of deguelin on premalignant HBE cells, the involvement of PI3K/Akt and MAPK pathways, which lead to increased cell proliferation or cell viability and are crucial for tumorigenesis (Sibilia et al., 2000; Franke et al., 1995) have been investigated. Numerous studies showed that the Akt pathway provides a critical cell survival signal for tumor progression by phosphorylation of a number of downstream proteins, including BAD, caspase-9, Forkhead transcription factors, IKK, Raf, and p21-activated protein kinase (Kobayashi et al., 1999; Moore et al., 1998).

The effects of deguelin on PI3K/Akt and MAPK activity in 1799 cells were next examined. The results from lipid kinase assay indicated that treatment of the 1799 cells with 10⁻⁷ M deguelin for 1 day decreased PI3K activity without changing the protein levels of PI3K components p85α and p110α. MAPK activity was not affected by the treatment of deguelin in the 1799 cells, suggesting that deguelin suppresses activation of the PI3K/Akt pathway in 1799 cells. Activation of the PI3K pathway generally causes selective phosphorylation of downstream effectors, such as Akt at Ser473/Thr308, GSK-3α/β at Ser9/21, and FKHR at Thr241/Ser256/Ser3l9 (Grimberg et al., 2000); therefore, the levels of pAkt (Ser473) and pGSK-3β (Ser9) were also examined by western blot analysis. The levels of pAkt (Ser473) and pGSK-3β (Ser9) were decreased in 1799 treated with deguelin in a time-dependent manner, whereas Akt, GSK-3α/β and β-Actin expression levels were not affected. Downregulation of the pAkt level by deguelin was correlated with the phosphorylation of the endogenous Akt-kinase substrate GSK-3β. Interestingly, the pAkt level was reduced after 7 h of treatment and was virtually undetectable after 14 h, although the activity of PI3K remained unaltered 14 h post-treatment and was suppressed after 24 h of treatment, suggesting that deguelin inhibits Akt activity through more than one pathway, including the inhibition of PB3K activation.

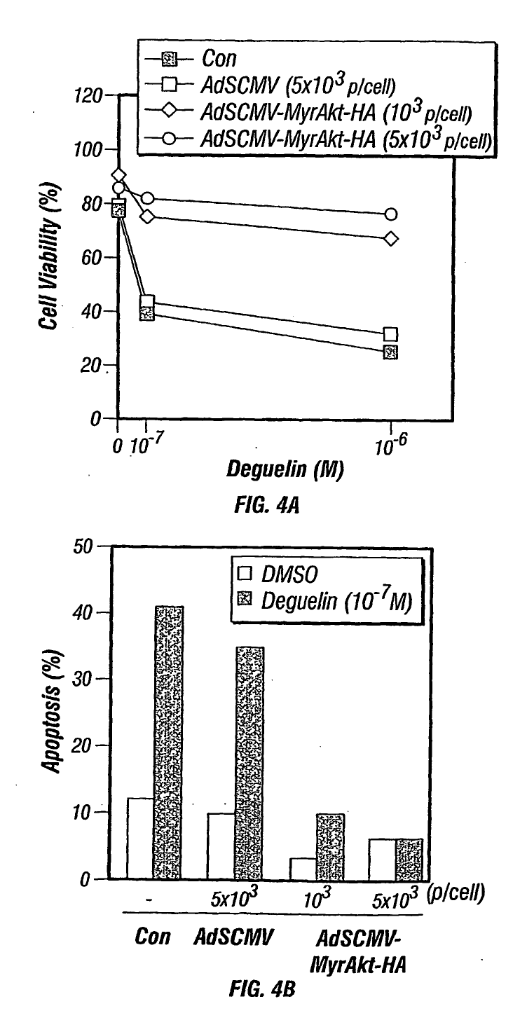

Protection of Deguelin-induced HBE Cells Death by Activation of PI3K/Akt in Premalignant. To confirm the premise that deguelin-induced apoptosis was mediated through the inhibition of PI3K/Akt activation, an adenovirus expressing an activated form of Akt with Src myristoylation signal fused in-frame to the c-Akt coding sequence with HA (Ad5CMV-Myr.Akt.HA) was constructed. To examine the induction of Myr.Akt.HA expression by Ad5CMV-Myr.Akt.HA, 1799 cells were infected with indicated titers of either empty virus (Ad5CMV) or virus expressing constitutively active Akt (Ad5CMV-Myr.Akt.HA) for 3 days, based on previous report. A time course of gene induction in HBE cells by adenoviral vector under the control of CMV promoter showed target gene expression beginning at 1 day, maximal expression at day 3, and rapid decrease after day 5 (Lee et al., 2002). Western blot analysis exhibited that Ad5CMV-Myr.Akt-HA induces a dose-dependent increase in the expression of HA and Myr.Akt.HA, which displayed a reduced mobility relative to Akt due to HA tag with no change in endogenous Akt. The activity of Ad5CMV-Myr.Akt.HA in the 1799 cells was verified by western blot analysis on pGSK-3β, a downstream effector of Akt. Accordingly, the 1799 cells were infected with increased doses of Ad5CMV-Myr.Akt.HA and then were tested for susceptibility to treatment with 10⁻⁷ M or 10⁻⁶ M deguelin. 1799 cells infected with Ad5CMV-Myr.Akt.HA showed a viral dose-dependent increase in cell survival in response to deguelin treatment (FIG. 4A). More than 80% of viable cells were observed in 1799 cells treated with 10⁻⁷ M of deguelin that were infected with 5×10³ M particles/cell of AdSCMV-Myr.Akt.HA, and even 10⁻⁶ M deguelin did not decrease the viable cell number. The empty virus (Ad5CMV) did not rescue 1799 cells from deguelin-mediated cell death. To determine whether the recovery of cell viability by Ad5CMV-Myr.Akt.HA was a result of protection from deguelin-induced apoptosis, 1799 cells infected with Ad5CMV-Myr.Akt.HA and then treated with deguelin as described above were collected and tested for susceptibility to the deguelin-induced apoptosis using the APO-BRDU staining kit. About 40% of untreated or empty virus-infected cells showed induction of apoptosis by 10⁻⁷ M of deguelin (FIG. 4B) compared with less than 10% of Ad5CMV-Myr.Akt.HA-infected 1799 cells, suggesting that the induction of apoptosis by deguelin in 1799 cells is due in part to inhibition of the PI3K/Akt-mediated antiapoptotic pathways. Taken together, these results suggested a crucial role of PI3K/Akt in deguelin-induced apoptosis.

Effects of Deguelin on Squamous Differentiated HBE Cells. Evidence is provided that PI3K is activated upon adenovirus interaction with αᵥ integrins and that this event is required for adenovirus internalization (Li et al., 1998). The premalignant and malignant cell lines used in this study were derived from an HBE cell immortalized with a hybrid adenovirus/simian virus 40. The phosphorylation of Akt in squamous HBE cells that mimic bronchial metaplasia, a potentially premalignant lesion induced in smokers (Lee et al., 1996) was investigated, to confirm whether the increase in. pAkt level in 1799 cells account for the premalignant stage of HBE cells. In tissue cultures, squamous HBE cells can be induced by growing HBE cells on tissue culture plates coated with a thin matrix of fibronectin and collagen or by the treatment with interferon (IFN)-r, transforming growth factor (TGF)-P, or phorbol esters (Jetten et al., 1986), and treatment with all-trans-retinoic acid, a known chemopreventive agent, inhibits this process (Lee et al., 1996). Prior studies demonstrated the increased expression of transglutaminase, involucrin, K5, and K13 in squamous HBE cells (Lee et al., 1996). After the induced expression of squamous marker genes, such as transglutaminase (TGase) and involucrin (Invol), was confirmed by northern blot analysis, western blot analysis on pAkt and pGSK-3β was performed to examine the activation of PI3K/Akt in squamous HBE cells. The level of pAkt and pGSK-3β was markedly induced in squamous HBE cells (S) compared to NHBE cells (N), whereas the expression of Akt and GSK-3α/β was same, indicating the activation of Akt in squamous HBE cells. It was then determined whether deguelin inhibits the activation of PI3K/Akt pathway in squamous HBE cells. The elevated levels of pAkt (Ser473) and pGSK-3β (Ser9) observed in squamous HBE cells were down-regulated by deguelin in a time-dependent manner. The apoptotic effects of deguelin and the involvement of PI3K/Akt pathway in squamous HBE cells were examined. After pronounced morphologic changes were observed in squamous HBE cells treated with deguelin at a concentration range of 10⁻⁹ M to 10⁻⁷ M for 1 day, the deguelin-mediated apoptosis was identified by examining reduction in the inactive form of caspase-3 and increase in PARP cleavage. Whether deguelin induces apoptosis in squamous HBE cells by suppressing the PI3K/Akt pathway was examined. For this study, squamous HBE cells infected with either Ad5CMV or Ad5CMV-Myr.Akt-HA were treated with with 10⁻⁷ M or 10⁻⁶ M of deguelin. Squamous HBE cells were protected from deguelin-induced cell death by the overexpression of constitutively active Akt, which was consistent with the results from 1799 cells treated with deguelin in similar conditions. According to western blot analysis, 1799 cells infected with Ad5CMV-Myr.Akt.HA showed increased level in the 32-kDa caspase-3 proenzyme accompanied by a decrease in the 89-kDa fragment. These findings indicated that deguelin induces apoptosis in squamous HBE cells by inhibiting PI3K/Akt pathway,

Akt was found to be constitutively active in premalignant and malignant HBE cells compared to NHBE cells. The activity of Akt is higher in 1799 cells (an immortalized HBE cell line) than in 1198 cells (an immortalized HBE cells exposed to carcinogen) or in 1170 cells (a malignant HBE cells). It has been demonstrated that overexpression of Akt is an early event during sporadic colon carcinogenesis (Phillips et al., 1998). In additioni, increased expression and/or activation of Akt have been observed in normal OSE from women with BRCA mutations (Shayesteh et al., 1999) and premalignant mammary hyperplasia that has an increased risk of progressing to tumors (Strange et al., 2001).

These findings suggest that activation of Akt is a common feature in early stage during the multistep carcinogenesis. The data provided herein now provide evidence that deguelin is an optimal agent for attacking Akt as it selectively blocked the activation of Akt in 1799 cells. Consequently, overexpression of constitutively active Akt protected 1799 cells from deguelin- induced apoptosis, indicating that the inhibition of Akt by deguelin is the mechanism that mediates its apoptotic effects in 1799 HBE cells. A partial and delayed inhibition of PI3K activity compared to the inhibition of Akt activity was observed in response to deguelin, suggesting that there are more than one mechanism that mediate the suppression of Akt activity by deguelin. It has been demonstrated that Akt can be activated independent of PI3K and MAPK by Ca²⁺/calmodulin-dependent protein kinase kinase where the increase in the intracellular Ca²⁺ concentration promotes survival of some cultured neurons (Yano et al., 1998). It was also observed that treatment of deguelin inhibits PI3K/Akt activity in 1198 and 1170 cells, and that constitutive Akt rescued these cell lines from deguelin-mediated apoptosis. Nevertheless, higher Akt activity in 1799 cells compared to 1198 and 1170 cells might result in increased relative sensitivity of the 1799 cells to deguelin. Studies on whether this unique mechanism applies to other PI3K inhibitors were performed, and it was observed that LY294002, a representiative PI3K inhibitor that blocks ATP binding to p110c PI3K catalytic domain; displayed much weaker efficacy in growth inhibition of premalignant HBE cells than deguelin (unpublished data); LY294002 required more than 10 μM to induce detectable cell growth inhibition in premalignant and malignant HBE cells, and it showed significant cytotoxicity on NHBE cells unlike deguelin.

### Example 4

### Deguelin Regulates Expression of COX-2

Of the COX enzymes, COX-1 has been found to be constitutively expressed in cells and plays a role in normal cell metabolic functions. COX-2 on the other hand, is found to be induced and expressed in neoplastic growth. COX-2 has been found to be involved in the prevention of lung carcinogenesis and to be regulated by Akt. Thus, it was determined whether deguelin regulates the expression of COX-2 in lung cancer cells. Normal, premalignant, and malignant lung cancer cells, NHBE, 1799, 1198, and 1170, were treated with 1 nM, 10 nM, or 100 nM deguelin for 1 day and COX-1 and COX-2 expression were analyzed by northern blotting. COX-2 expression was observed to be higher in premalignant cells (HBE 1799, 1198 cells) compared to the malignant (HBE 1170) or normal cells.

It was also observed that induced COX-2 expression was downregulated by deguelin in the premalignant cells. In this study, the protein and mRNA expression of COX-1 and COX-2 were tested in 1799 and squamous (Sq) HBE cells. These cells were treated with 1 nM, 10 nM, or 100 nM deguelin and the COX-1 and COX-2 RNA and analyzed by northern blotting and western blotting. Equal amount of mRNA in each lane was confirmed by northern blot analysis using GAPDH (data not shown).

### Example 5

### Apoptotic Effect of Deguelin on HBE Cells

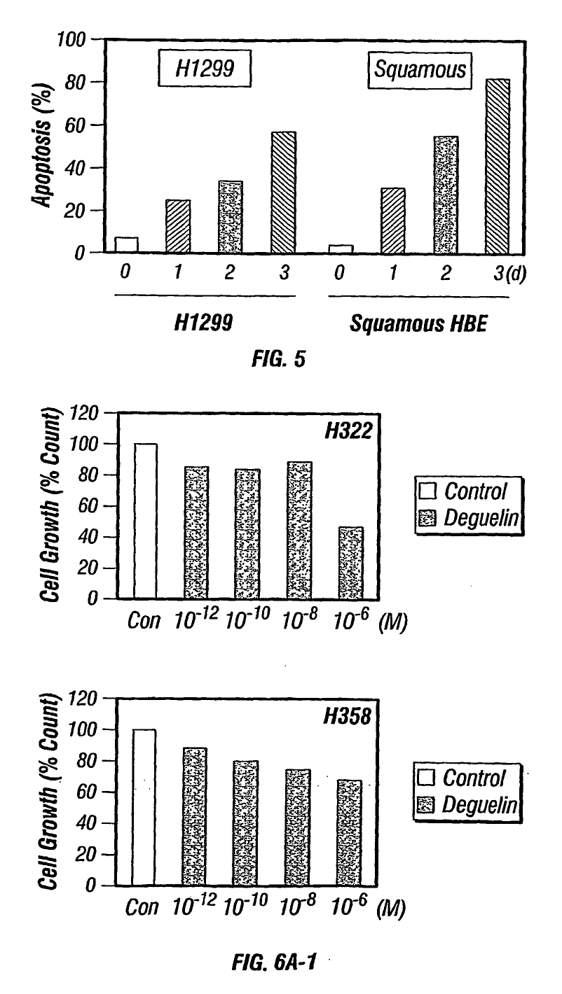

The apoptotic effect of deguelin was further assessed in HBE cells. Cells were treated with 10⁻⁷ M deguelin for 1, 2, or 3 days. Apoptosis was analyzed by flow cytometry as described above. All cell lines tested showed 60% or greater apoptosis by day 2 or day 3 as is demonstrated for H1299 and squamous HBE cells (FIG. 5). To confirm the apoptotic activity in these cells, bax and bcl-2 expression were analyzed by western blotting. Increased bax expression was observed in the cell lines and correlated with the apoptotic activity observed by FACS analysis. Thus, it was determined that deguelin increases bax expression thereby inducing the apoptotic activity in lung cancer cells. Bcl-2 expression was not found to be regulated in the presence of deguelin in the cells lines tested.

### Example 6

### Growth Inhibitory Effect of Deguelin and Deguelin Derivatives on NSCLC Cells

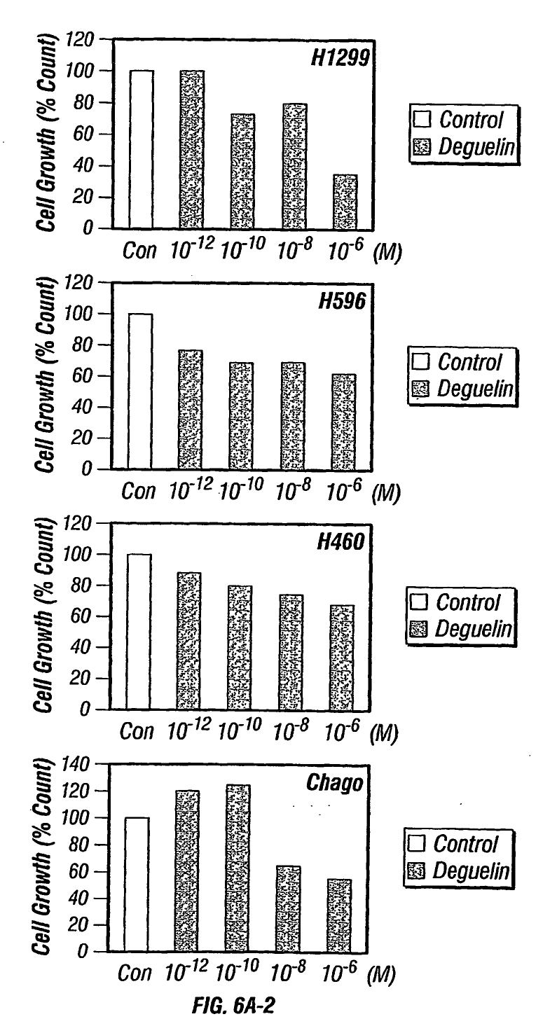

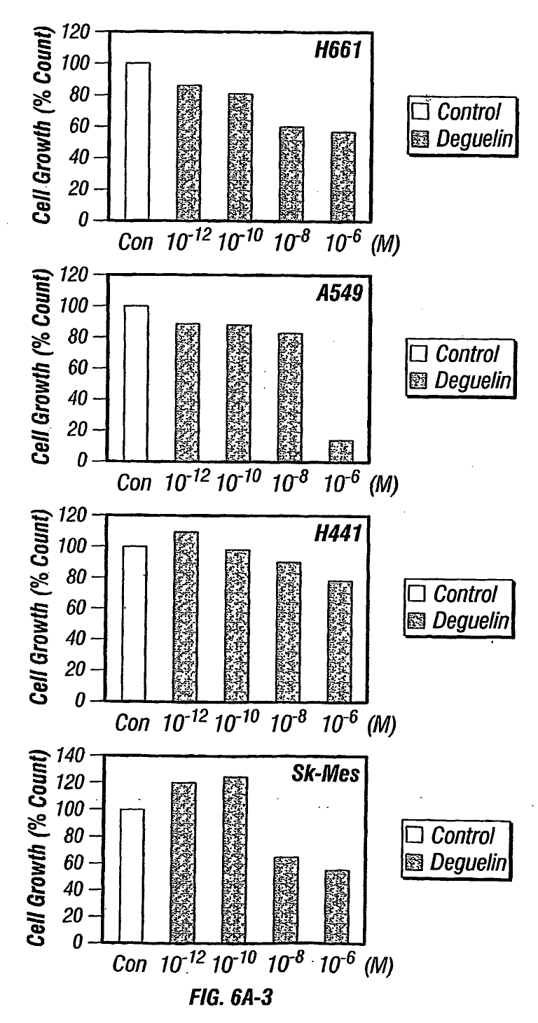

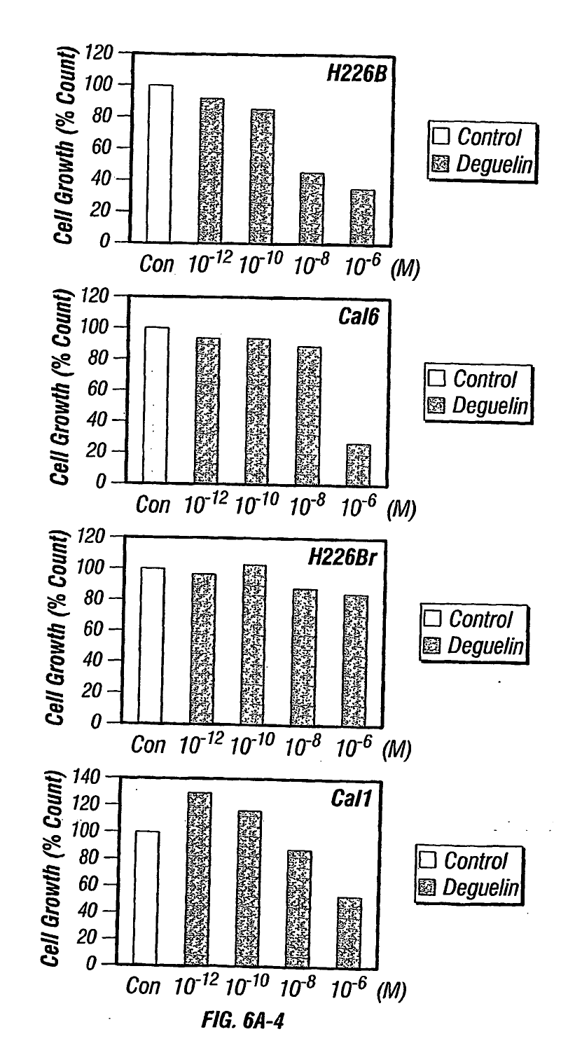

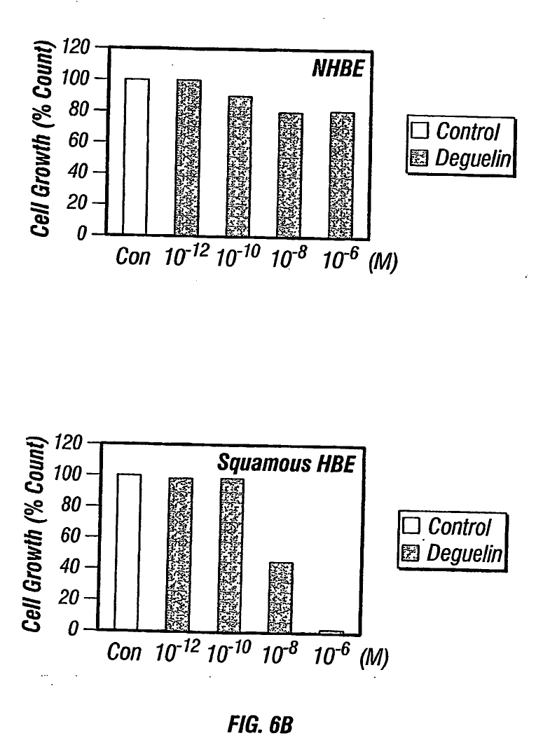

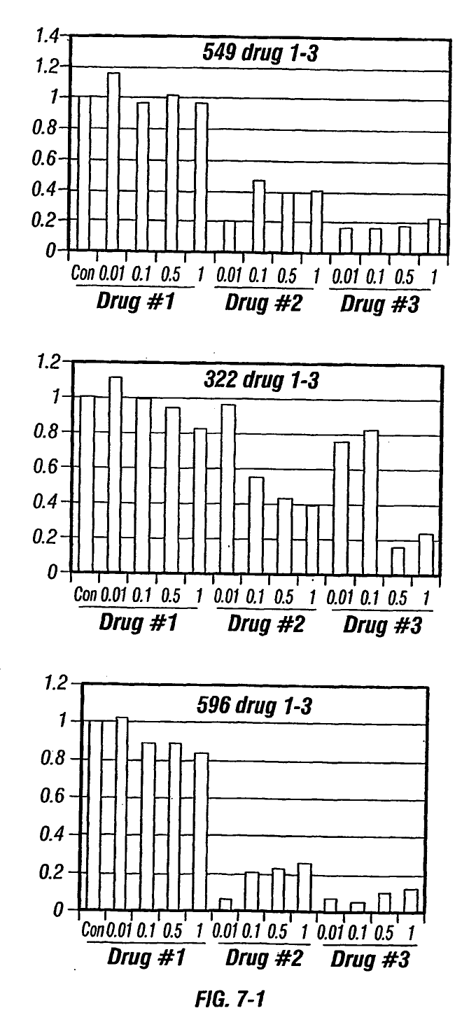

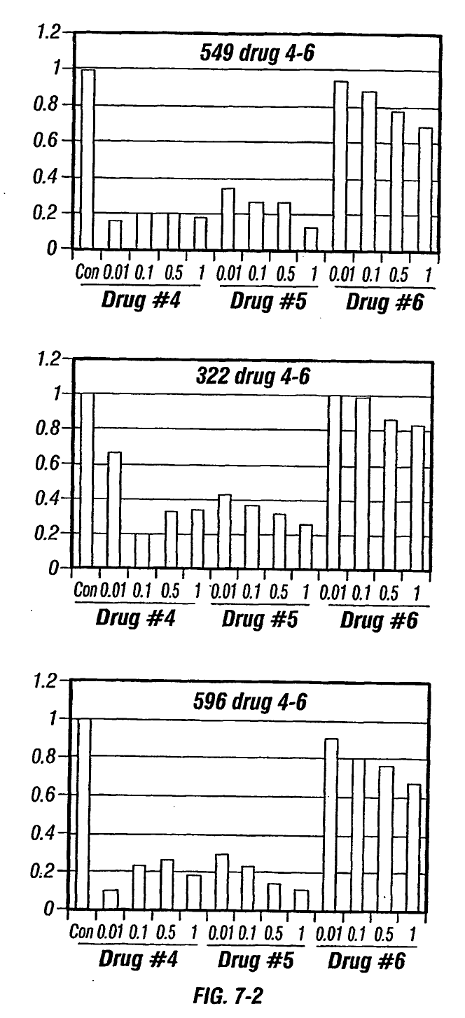

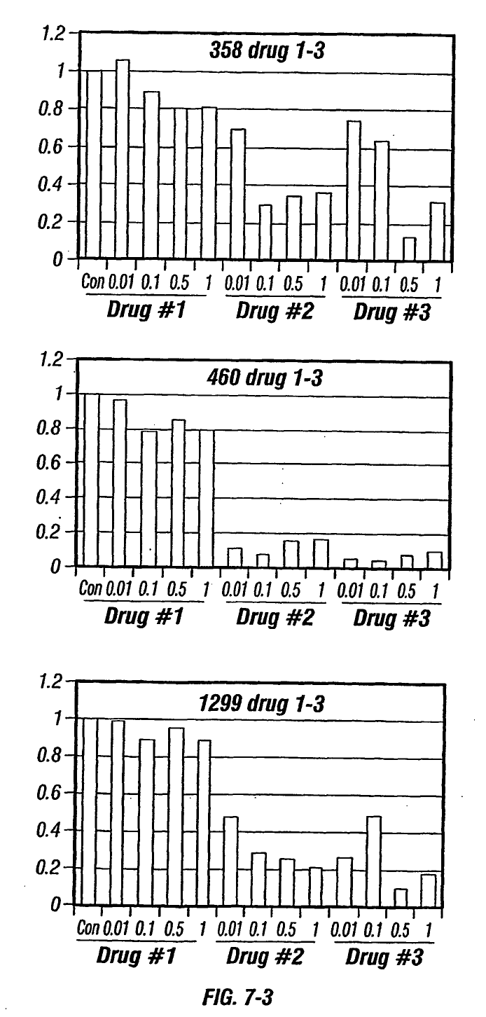

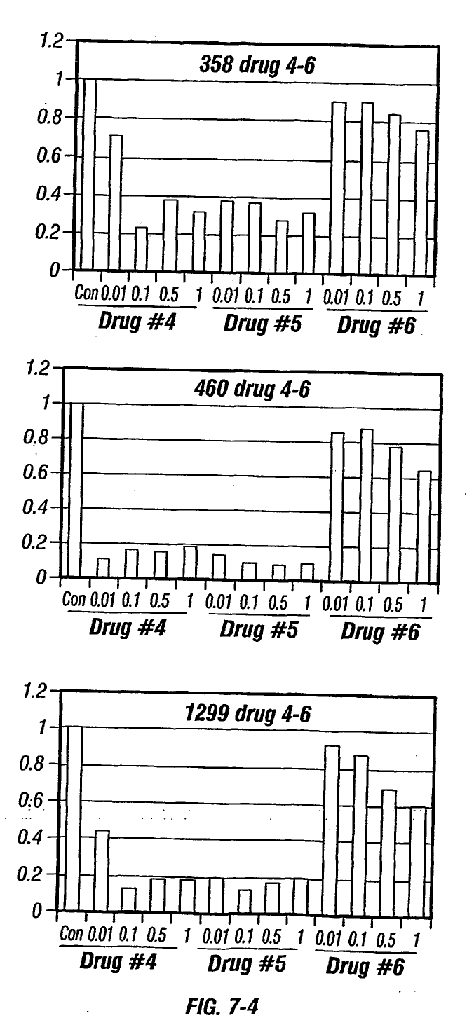

To further investigate the chemopreventive activity of deguelin, inhibition of cell proliferation was determined in normal, premalignant and malignant non-small cell lung cancer cells (NSCLC). Cells were transferred onto 96-well plates at densities ranging from 2×10³ to 4×10³ cells/well. After 1 day, the cells were changed to fresh medium containing various concentrations of deguelin or deguelin derivatives dissolved in DMSO (final concentration, 0.1%). Control cultures received 0.1% dimethyl sulfoxide (DMSO) as did the deguelin- or deguelin derivative-treated cultures. Cells were treated with 10⁻⁷ M or 10⁻⁶ M of deguelin; 0.01 μM, 0.1 μM, 0.5 μM, or 1 μM each of a deguelin derivative; or 0.1% DMSO as a control, and then incubated for 3 days. The viability of treated cells was measured by 3-(4,5-dimethylthiazol-2-yl)-2,5-diphenyltetrazolium bromide (MTT) assay as previously described (Lee et al., 2002). Six replicate wells were used for each analysis. The drug concentration required to cause 50% cell growth inhibition (IC₅₀) was determined by interpolation from dose-response curves. The deguelin derivatives used were: 6a,2a-dehydrorotenone; methoxyrot-2′-enoic acid; tephrosin; 7S-hydroxydeguelin; rotenone; 7a,13a-dehydrodeguelin; 12-hydroxyrotenone; 12,12a-dehydrorotenone; isorotenone; 4-chlororot-2′-enoic acid; 1,2-dihydrodeguelin; 2-phenylselenyl-1,2,-dihydrodeguelin; 2-phenylselenyl-1,2-dihydrodeguelin; and bromorot-2′-enoic acid. The IC₅₀ for deguelin was found to be 10⁻⁷ M to 10⁻⁶ M depending on the cell line. As shown in Table 1 and FIG. 6, most cells were sensitive to deguelin at 10⁻⁷ M to 10⁻⁶ M. The deguelin derivatives: methoxyrot-2′-enoic acid, tephrosin, 7S-hydroxydeguelin and bromorot-2′-enoic acid appeared to be the most effective of the compounds in inhibiting cell growth in NSCLC cells. Table 2; FIG. 7.

TABLE 1
Deguelin

| Cell | | | | | | Cell | | | | | |
|---|---|---|---|---|---|---|---|---|---|---|---|
| Lines | Control | 10⁻¹²M | 10⁻¹⁰M | 10⁻⁸M | 10⁻⁶M | Lines | Control | 10⁻¹²M | 10⁻¹⁰M | 10⁻⁸M | 10⁻⁶M |
| H1299 | 0.147 | 0.121 | 0.119 | 0.101 | 0.052 | H661 | 0.12 | 0.09 | 0.087 | 0.062 | 0.059 |
| | 0.13 | 0.136 | 0.087 | 0.108 | 0.043 | | 0.092 | 0.084 | 0.093 | 0.056 | 0.055 |
| | 0.125 | 0.138 | 0.089 | 0.103 | 0.049 | | 0.099 | 0.094 | 0.07 | 0.07 | 0.065 |
| | 0.134 | 0.132 | 0.098 | 0.104 | 0.048 | | 0.104 | 0.089 | 0.083 | 0.063 | 0.0597 |
| Avg. % con | 1 | 0.98 | 0.73 | 0.78 | 0.36 | Avg. % con | 1 | 0.86 | 0.80 | 0.60 | 0.5756 |
| H596 | 0.426 | 0.386 | 0.314 | 0.324 | 0.29 | A549 | 0.476 | 0.416 | 0.375 | 0.381 | 0.057 |
| | 0.491 | 0.372 | 0.338 | 0.311 | 0.298 | | 0.462 | 0.404 | 0.422 | 0.381 | 0.057 |
| | 0.494 | 0.317 | 0.287 | 0.307 | 0.281 | | 0.451 | 0.412 | 0.427 | 0.385 | 0.066 |
| | 0.470 | 0.358 | 0.313 | 0.314 | 0.290 | | 0.463 | 0.411 | 0.408 | 0.382 | 0.060 |
| Avg. % con | 1 | 0.76 | 0.67 | 0.67 | 0.62 | Avg. % con | 1 | 0.89 | 0.88 | 0.83 | 0.13 |
| H460 | 0.773 | 0.797 | 0.783 | 0.632 | 0.115 | H441 | 0.227 | 0.234 | 0.21 | 0.209 | 0.18 |
| | 0.733 | 0.75 | 0.761 | 0.652 | 0.141 | | 0.217 | 0.259 | 0.231 | 0.203 | 0.18 |
| | 0.72 | 0.816 | 0.743 | 0.62 | 0.131 | | 0.22 | 0.249 | 0.229 | 0.21 | 0.183 |
| | 0.742 | 0.788 | 0.762 | 0.635 | 0.129 | | 0.221 | 0.247 | 0.223 | 0.207 | 0.181 |
| Avg. % con | 1 | 1.06 | 1.03 | 0.86 | 0.17 | Avg. % con | 1 | 1.12 | 1.01 | 0.94 | 0.82 |
| H358 | 0.154 | 0.148 | 0.122 | 0.114 | 0.108 | H322 | 0.207 | 0.168 | 0.153 | 0.172 | 0.096 |
| | 0.161 | 0.143 | 0.12 | 0.128 | 0.113 | | 0.192 | 0.16 | 0.16 | 0.174 | 0.087 |
| | 0.178 | 0.139 | 0.146 | 0.125 | 0.112 | | 0.181 | 0.17 | 0.177 | 0.172 | 0.1 |
| | 0.164 | 0.143 | 0.129 | 0.122 | 0.111 | | 0.193 | 0.166 | 0.163 | 0.173 | 0.094 |
| Avg. % con | 1 | 0.87 | 0.79 | 0.74 | 0.68 | Avg. % con | 1 | 0.86 | 0.84 | 0.89 | 0.49 |
| H226B | 0.351 | 0.274 | 0.295 | 0.153 | 0.117 | H226Br | 0.325 | 0.3 | 0.321 | 0.296 | 0.264 |
| | 0.329 | 0.32 | 0.282 | 0.147 | 0.106 | | 0.326 | 0.318 | 0.329 | 0.273 | 0.264 |
| | 0.306 | 0.304 | 0.272 | 0.149 | 0.117 | | 0.319 | 0.313 | 0.34 | 0.259 | 0.271 |
| | 0.329 | 0.299 | 0.283 | 0.150 | 0.113 | | 0.323 | 0.310 | 0.330 | 0.276 | 0.266 |
| Avg. % con | 1 | 0.91 | 0.86 | 0.46 | 0.34 | Av. % con | 1 | 0.96 | 1.02 | 0.85 | 0.82 |
| Cal6 | 0.381 | 0.357 | 0.332 | 0.356 | 0.098 | Cal1 | 0.241 | 0.305 | 0.29 | 0.204 | 0.13 |
| | 0.396 | 0.358 | 0.386 | 0.338 | 0.106 | | 0.253 | 0.328 | 0.294 | 0.221 | 0.128 |
| | 0.4 | 0.366 | 0.374 | 0.341 | 0.078 | | 0.236 | 0.319 | 0.26 | 0.216 | 0.138 |
| | 0.392 | 0.360 | 0.364 | 0.345 | 0.094 | | 0.243 | 0.317 | 0.281 | 0.214 | 0.132 |
| Avg. % con | 1 | 0.92 | 0.93 | 0.88 | 0.24 | Avg. % con | 1 | 1.30 | 1.16 | 0.88 | 0.54 |
| Chago | 0.158 | 0.175 | 0.192 | 0.101 | 0.094 | Sk-mes | 0.279 | 0.253 | 0.243 | 0.207 | 0.107 |
| | 0.158 | 0.179 | 0.184 | 0.093 | 0.086 | | 0.268 | 0.258 | 0.223 | 0.209 | 0.135 |
| | 0.146 | 0.192 | 0.197 | 0.106 | 0.078 | | 0.258 | 0.264 | 0.274 | 0.216 | 0.122 |
| | 0.154 | 0.182 | 0.191 | 0.1 | 0.086 | | 0.268 | 0.258 | 0.247 | 0.211 | 0.121 |
| Avg. % con | 1 | 1.18 | 1.24 | 0.65 | 0.56 | Avg. % con | 1 | 0.96 | 0.92 | 0.79 | 0.45 |
| NHBE | 0.402 | 0.417 | 0.355 | 0.249 | 0.129 | | 0.396 | 0.392 | 0.337 | 0.288 | 0.306 |
| | 0.469 | 0.365 | 0.341 | 0.227 | 0.082 | | 0.37 | 0.386 | 0.327 | 0.291 | 0.299 |
| | 0.412 | 0.377 | 0.346 | 0.21 | 0.087 | | 0.358 | 0.367 | 0.343 | 0.283 | 0.305 |
| | | | | | | | 0.362 | 0.362 | 0.332 | 0.301 | 0.306 |
| | 0.428 | 0.386 | 0.347 | 0.229 | 0.099 | | 0.3715 | 0.3768 | 0.3348 | 0.2908 | 0.304 |
| Avg. % con | 1 | 0.90 | 0.81 | 0.53 | 0.23 | Avg. % con | 1 | 1.01 | 0.90 | 0.78 | 0.82 |

TABLE 2

| | | 6a,2a-dehydrorotenone | | | | Methoxyrot-2′-enoic acid | | | | Tephrosin | | | |
|---|---|---|---|---|---|---|---|---|---|---|---|---|---|
| | | (Drug 1) | | | | (Drug 2) | | | | (Drug 3) | | | |
| | Control | 0.01 μM | 0.1 μM | 0.5 μM | 1 μM | 0.01 μM | 0.1 μM | 0.5 μM | 1 μM | 0.01 μM | 0.1 μM | 0.5 μM | 1 μM |
| H1299 | 1.544 | 1.63 | 1.539 | 1.502 | 1.57 | 0.793 | 0.387 | 0.497 | 0.301 | 0.399 | 0.784 | 0.204 | 0.26 |
| | 1.342 | 1.515 | 1.281 | 1.595 | 1.421 | 0.86 | 0.526 | 0.278 | 0.289 | 0.471 | 0.956 | 0.179 | 0.36 |
| | 1.643 | 1.517 | 1.351 | 1.413 | 1.33 | 0.656 | 0.465 | 0.503 | 0.346 | 0.527 | 0.491 | 0.097 | 0.183 |
| | 1.749 | 1.516 | 1.481 | 1.503 | 1.446 | 0.718 | 0.518 | 0.304 | 0.372 | 0.447 | 0.807 | 0.127 | 0.319 |
| | 1.551 | 1.562 | 1.39 | 1.544 | 1.452 | 0.707 | 0.401 | 0.425 | 0.29 | 0.38 | 0.795 | 0.135 | 0.261 |
| | 1.555 | 1.561 | 1.308 | 1.561 | 1.321 | 0.725 | 0.474 | 0.405 | 0.384 | 0.248 | 0.806 | 0.147 | 0.279 |
| | 1.595 | 1.559 | 1.385 | 1.338 | 1.233 | 0.796 | 0.434 | 0.342 | 0.395 | 0.428 | 0.783 | 0.166 | 0.255 |
| Average | 1.5684 | 1.5514 | 1.3907 | 1.4937 | 1.3961 | 0.7507 | 0.4579 | 0.3934 | 0.3396 | 0.4143 | 0.7746 | 0.1507 | 0.2739 |
| SD | 0.1233 | 0.0412 | 0.0919 | 0.0897 | 0.1104 | 0.0688 | 0.0538 | 0.0893 | 0.0459 | 0.0878 | 0.139 | 0.0355 | 0.0555 |
| SD/2 | 0.0617 | 0.0206 | 0.0459 | 0.0448 | 0.0552 | 0.0344 | 0.0269 | 0.0446 | 0.023 | 0.0439 | 0.0695 | 0.0178 | 0.0277 |
| % control | 1 | 0.99 | 0.89 | 0.95 | 0.89 | 0.48 | 0.29 | 0.25 | 0.22 | 0.26 | 0.49 | 0.10 | 0.17 |
| A549 | 1.288 | 1.69 | 0.691 | 1.531 | 1.544 | 0.286 | 0.693 | 0.542 | 0.783 | 0.274 | 0.161 | 0.299 | 0.434 |
| | 1.268 | 1.705 | 1.805 | 1.925 | 1.458 | 0.219 | 0.467 | 0.837 | 0.583 | 0.196 | 0.279 | 0.243 | 0.15 |
| | 1.331 | 1.541 | 1.719 | 1.594 | 1.375 | 0.272 | 0.636 | 0.514 | 0.67 | 0.223 | 0.299 | 0.26 | 0.366 |
| | 1.399 | 1.69 | 1.454 | 1.387 | 1.486 | 0.298 | 0.687 | 0.534 | 0.758 | 0.284 | 0.218 | 0.217 | 0.295 |
| | 1.549 | 1.829 | 1.375 | 1.359 | 1.428 | 0.36 | 0.753 | 0.588 | 0.515 | 0.229 | 0.262 | 0.217 | 0.386 |
| | 1.575 | 1.786 | 1.494 | 1.356 | 1.464 | 0.344 | 0.796 | 0.579 | 0.477 | 0.252 | 0.236 | 0.305 | 0.341 |
| | 1.568 | 1.706 | 1.375 | 1.258 | 1.242 | 0.309 | 0.751 | 0.462 | 0.436 | 0.224 | 0.281 | 0.244 | 0.347 |
| Average | 1.4679 | 1.7067 | 1.4161 | 1.4871 | 1.4281 | 0.2983 | 0.6833 | 0.5794 | 0.6031 | 0.2403 | 0.248 | 0.255 | 0.3313 |
| SD | 0.1741 | 0.0906 | 0.3605 | 0.2241 | 0.097 | 0.0468 | 0.109 | 0.1211 | 0.1371 | 0.0312 | 0.0474 | 0.0356 | 0.0906 |
| SD/2 | 0.087 | 0.0453 | 0.1802 | 0.112 | 0.0485 | 0.0234 | 0.0545 | 0.0605 | 0.0686 | 0.0156 | 0.0237 | 0.0178 | 0.0453 |
| % control | 1 | 1.16 | 0.96 | 1.01 | 0.97 | 0.20 | 0.47 | 0.39 | 0.41 | 0.16 | 0.17 | 0.17 | 0.23 |
| H322 | 0.916 | 0.795 | 0.865 | 0.736 | 0.677 | 0.729 | 0.322 | 0.32 | 0.271 | 0.527 | 0.685 | 0.113 | 0.223 |
| | 0.952 | 0.799 | 0.771 | 0.809 | 0.658 | 0.806 | 0.412 | 0.314 | 0.348 | 0.596 | 0.659 | 0.109 | 0.216 |
| | 0.863 | 0.893 | 0.799 | 0.833 | 0.692 | 0.748 | 0.406 | 0.388 | 0.409 | 0.713 | 0.726 | 0.109 | 0.182 |
| | 0.811 | 0.989 | 0.872 | 0.746 | 0.681 | 0.791 | 0.52 | 0.393 | 0.352 | 0.655 | 0.643 | 0.12 | 0.168 |
| | 0.881 | 1.014 | 0.866 | 0.89 | 0.68 | 0.898 | 0.585 | 0.466 | 0.361 | 0.68 | 0.732 | 0.117 | 0.216 |
| | 0.889 | 1.101 | 0.909 | 0.958 | 0.859 | 1.011 | 0.58 | 0.443 | 0.324 | 0.721 | 0.795 | 0.162 | 0.241 |
| | 0.848 | 1.192 | 0.977 | 0.824 | 0.802 | 0.921 | 0.537 | 0.327 | 0.358 | 0.747 | 0.798 | 0.21 | 0.216 |
| Average | 0.8738 | 0.969 | 0.8656 | 0.828 | 0.7213 | 0.8434 | 0.4803 | 0.3787 | 0.3461 | 0.6627 | 0.7197 | 0.1343 | 0.2089 |
| SD | 0.0496 | 0.1497 | 0.068 | 0.0778 | 0.0771 | 0.1028 | 0.1008 | 0.061 | 0.0418 | 0.0777 | 0.0616 | 0.0382 | 0.0251 |
| SD/2 | 0.0248 | 0.0749 | 0.034 | 0.0389 | 0.0385 | 0.0514 | 0.0504 | 0.0305 | 0.0209 | 0.0389 | 0.0308 | 0.0191 | 0.0125 |
| % control | 1 | 1.11 | 0.99 | 0.95 | 0.83 | 0.97 | 0.55 | 0.43 | 0.40 | 0.76 | 0.82 | 0.15 | 0.24 |
| H596 | 1.696 | 1.581 | 1.347 | 1.343 | 1.234 | | 0.287 | 0.28 | 0.422 | 0.062 | 0.082 | 0.137 | 0.287 |
| | 1.575 | 1.597 | 1.381 | 1.467 | 1.257 | 0.037 | 0.3 | 0.434 | 0.37 | 0.127 | 0.062 | 0.159 | 0.257 |
| | 1.553 | 1.651 | 1.333 | 1.312 | 1.341 | 0.055 | 0.326 | 0.344 | 0.481 | 0.078 | 0.066 | 0.151 | 0.172 |
| | 1.549 | 1.605 | 1.513 | 1.463 | 1.494 | 0.067 | 0.366 | 0.446 | 0.573 | 0.138 | 0.095 | 0.111 | 0.151 |
| | 1.619 | 1.631 | 1.417 | 1.351 | 1.326 | 0.122 | 0.289 | 0.348 | 0.319 | 0.103 | 0.09 | 0.158 | 0.215 |
| | 1.499 | 1.617 | 1.34 | 1.399 | 1.319 | 0.101 | 0.321 | 0.383 | 0.328 | 0.138 | 0.058 | 0.076 | 0.131 |
| | 1.586 | 1.58 | 1.462 | 1.476 | 1.279 | 0.118 | 0.338 | 0.322 | 0.342 | 0.163 | 0.125 | 0.35 | 0.175 |
| Average | 1.5701 | 1.6089 | 1.399 | 1.4016 | 1.3214 | 0.0931 | 0.3181 | 0.3653 | 0.405 | 0.1156 | 0.0826 | 0.1631 | 0.1983 |
| SD | 0.085 | 0.0261 | 0.0685 | 0.0678 | 0.0854 | 0.0414 | 0.0286 | 0.0598 | 0.0938 | 0.0361 | 0.0235 | 0.0877 | 0.0571 |
| SD/2 | 0.0425 | 0.0131 | 0.0343 | 0.0339 | 0.0427 | 0.0207 | 0.0143 | 0.0299 | 0.0469 | 0.0181 | 0.0117 | 0.0438 | 0.0286 |
| % control | 1 | 1.02 | 0.89 | 0.89 | 0.84 | 0.06 | 0.20 | 0.23 | 0.26 | 0.07 | 0.05 | 0.10 | 0.13 |
| H358 | 0.852 | 0.784 | 0.602 | 0.533 | 0.686 | 0.414 | 0.196 | 0.212 | 0.283 | 0.508 | 0.522 | 0.054 | 0.245 |
| | 0.873 | 0.734 | 0.652 | 0.604 | 0.624 | 0.525 | 0.173 | 0.331 | 0.248 | 0.601 | 0.496 | 0.048 | 0.233 |
| | 0.83 | 0.786 | 0.603 | 0.6 | 0.639 | 0.579 | 0.287 | 0.284 | 0.248 | 0.658 | 0.558 | 0.1 | 0.332 |
| | 0.81 | 0.686 | 0.672 | 0.736 | 0.64 | 0.603 | 0.284 | 0.282 | 0.306 | 0.674 | 0.496 | 0.105 | 0.139 |
| | 0.745 | 0.95 | 0.792 | 0.633 | 0.667 | 0.68 | 0.293 | 0.478 | 0.3 | 0.608 | 0.516 | 0.117 | 0.234 |
| | 0.928 | 1.192 | 0.778 | 0.734 | 0.752 | 0.612 | 0.247 | 0.2 | 0.343 | 0.59 | 0.579 | 0.147 | 0.293 |
| | 0.873 | 1.071 | 1.159 | 0.889 | 0.78 | 0.65 | 0.223 | 0.232 | 0.405 | 0.705 | 0.562 | 0.162 | 0.368 |
| Average | 0.8422 | 0.8861 | 0.7511 | 0.6756 | 0.684 | 0.5804 | 0.2433 | 0.2884 | 0.3047 | 0.6206 | 0.5327 | 0.1047 | 0.2634 |
| SD | 0.0511 | 0.1895 | 0.1954 | 0.1194 | 0.0601 | 0.0885 | 0.0477 | 0.0954 | 0.0555 | 0.0652 | 0.0335 | 0.0429 | 0.0754 |
| SD/2 | 0.0255 | 0.0948 | 0.0977 | 0.0597 | 0.0301 | 0.0443 | 0.0238 | 0.0477 | 0.0277 | 0.0326 | 0.0167 | 0.0214 | 0.0377 |
| % control | 1 | 1.05 | 0.89 | 0.80 | 0.81 | 0.69 | 0.29 | 0.34 | 0.36 | 0.74 | 0.63 | 0.12 | 0.31 |
| H460 | 1.746 | 1.625 | 1.314 | 1.31 | 1.253 | 0.037 | 0.139 | 0.297 | 0.276 | 0.072 | 0.072 | 0.088 | 0.162 |
| | 1.706 | 1.563 | 1.314 | 1.421 | 1.353 | 0.62 | 0.105 | 0.26 | 0.322 | 0.051 | 0.067 | 0.113 | 0.184 |
| | 1.684 | 1.731 | 1.333 | 1.538 | 1.351 | 0.096 | 0.129 | 0.302 | 0.282 | 0.097 | 0.086 | 0.113 | 0.154 |
| | 1.765 | 1.89 | 1.204 | 1.504 | 1.347 | 0.098 | 0.16 | 0.283 | 0.298 | 0.1 | 0.112 | 0.159 | 0.272 |
| | 1.818 | 1.443 | 1.501 | 1.55 | 1.429 | 0.122 | 0.133 | 0.291 | 0.311 | 0.105 | 0.056 | 0.16 | 0.183 |
| | 1.752 | 1.675 | 1.379 | 1.534 | 1.319 | 0.16 | 0.147 | 0.282 | 0.29 | 0.132 | 0.092 | 0.19 | 0.133 |
| | 1.722 | 1.815 | 1.521 | 1.573 | 1.622 | 0.195 | 0.166 | 0.235 | 0.274 | 0.112 | 0.086 | 0.164 | 0.185 |
| Average | 1.7463 | 1.6774 | 1.3666 | 1.49 | 1.382 | 0.1897 | 0.1399 | 0.2786 | 0.2933 | 0.0956 | 0.0816 | 0.141 | 0.1819 |
| SD | 0.0414 | 0.1515 | 0.1119 | 0.0932 | 0.1179 | 0.1963 | 0.0205 | 0.0235 | 0.0181 | 0.0266 | 0.0184 | 0.0365 | 0.0442 |
| SD/2 | 0.0207 | 0.0758 | 0.056 | 0.0466 | 0.059 | 0.0981 | 0.0102 | 0.0118 | 0.0091 | 0.0133 | 0.0092 | 0.0182 | 0.0221 |
| % control | 1 | 0.96 | 0.78 | 0.85 | 0.79 | 0.11 | 0.08 | 0.16 | 0.17 | 0.05 | 0.05 | 0.08 | 0.10 |
| | | 7s-Hydroxydeguelin | | | | Rotenone | | | | 7a,13a-dehydrodeguelin | | | |
| | | (Drug 4) | | | | (Drug 5) | | | | (Drug 6) | | | |
| | Control | 0.01 μM | 0.1 μM | 0.5 μM | 1 μM | 0.01 μM | 0.1 μM | 0.5 μM | 1 μM | 0.01 μM | 0.1 μM | 0.5 μM | 1 μM |
| H1299 | 1.379 | 0.565 | 0.109 | 0.342 | 0.235 | 0.249 | 0.25 | 0.308 | 0.226 | 1.311 | 1.228 | 1.033 | 0.769 |
| | 1.472 | 0.621 | 0.147 | 0.248 | 0.3 | 0.392 | 0.3 | 0.259 | 0.193 | 1.344 | 1.331 | 1.017 | 0.838 |
| | 1.379 | 0.503 | 0.102 | 0.129 | 0.258 | 0.221 | 0.158 | 0.248 | 0.23 | 1.402 | 1.318 | 1.106 | 0.938 |
| | 1.476 | 0.581 | 0.146 | 0.21 | 0.22 | 0.209 | 0.09 | 0.197 | 0.175 | 1.302 | 1.141 | 0.913 | 0.813 |
| | 1.424 | 0.711 | 0.248 | 0.272 | 0.243 | 0.261 | 0.125 | 0.195 | 0.76 | 1.286 | 1.196 | 0.874 | 0.837 |
| | 1.263 | 0.755 | 0.268 | 0.273 | 0.252 | 0.311 | 0.235 | 0.179 | 0.148 | 1.293 | 1.153 | 0.947 | |
| | 1.436 | 0.723 | 0.271 | 0.312 | 0.242 | 0.313 | 0.177 | 0.288 | 0.195 | 1.287 | 1.295 | 0.905 | 0.876 |
| Average | 1.4156 | 0.637 | 0.1844 | 0.2551 | 0.25 | 0.2794 | 0.1907 | 0.2391 | 0.2753 | 1.3179 | 1.2374 | 0.9707 | 0.8452 |
| SD | 0.0696 | 0.0943 | 0.0751 | 0.07 | 0.0252 | 0.0638 | 0.0743 | 0.0499 | 0.2156 | 0.0421 | 0.0783 | 0.0836 | 0.0575 |
| SD/2 | 0.0348 | 0.0471 | 0.0376 | 0.035 | 0.0126 | 0.0319 | 0.0372 | 0.025 | 0.1078 | 0.0211 | 0.0392 | 0.0418 | 0.0287 |
| % control | 1 | 0.45 | 0.13 | 0.18 | 0.18 | 0.20 | 0.13 | 0.17 | 0.19 | 0.93 | 0.87 | 0.69 | 0.60 |
| A549 | 1.43 | 0.155 | 0.284 | 0.184 | 0.254 | 0.425 | 0.418 | 0.284 | 0.188 | 1.182 | 1.073 | 1.107 | 0.97 |
| | 1.379 | 0.225 | 0.26 | 0.23 | 0.207 | 0.439 | 0.293 | 0.833 | 0.171 | 1.147 | 1.17 | 1.032 | 0.971 |
| | 1.374 | 0.173 | 0.245 | 0.257 | 0.221 | 0.541 | 0.364 | 0.266 | 0.176 | 1.307 | 1.208 | 0.931 | 1.014 |
| | 1.353 | 0.237 | 0.293 | 0.246 | 0.263 | 0.451 | 0.26 | 0.314 | 0.213 | 1.208 | 1.308 | 0.964 | 0.89 |
| | 1.359 | 0.226 | 0.256 | 0.251 | 0.253 | 0.45 | 0.429 | 0.246 | 0.173 | 1.279 | 1.211 | 1.036 | 0.862 |
| | 1.123 | 0.229 | 0.251 | 0.41 | 0.24 | 0.428 | 0.354 | 0.308 | 0.141 | 1.361 | 1.152 | 1.104 | 0.886 |
| | 1.238 | 0.26 | 0.3 | 0.31 | 0.268 | 0.507 | 0.439 | 0.309 | 0.192 | 1.334 | 1.149 | 1.121 | 0.863 |
| Average | 1.3332 | 0.215 | 0.2699 | 0.2697 | 0.2437 | 0.463 | 0.3653 | 0.3657 | 0.1791 | 1.2597 | 1.1816 | 1.0421 | 0.9223 |
| SD | 0.0955 | 0.0372 | 0.022 | 0.0722 | 0.0225 | 0.0439 | 0.0692 | 0.2076 | 0.0222 | 0.0814 | 0.0723 | 0.074 | 0.0613 |
| SD/2 | 0.0477 | 0.0186 | 0.011 | 0.0361 | 0.0112 | 0.022 | 0.0346 | 0.1038 | 0.0111 | 0.0407 | 0.0362 | 0.037 | 0.0307 |
| % control | 1 | 0.16 | 0.20 | 0.20 | 0.18 | 0.35 | 0.27 | 0.27 | 0.13 | 0.94 | 0.89 | 0.78 | 0.69 |
| H322 | 1.014 | 0.548 | 0.164 | 0.311 | 0.368 | 0.426 | 0.339 | 0.286 | 0.254 | 1.142 | 1.013 | 0.907 | 0.969 |
| | 0.987 | 0.54 | 0.118 | 0.381 | 0.314 | 0.477 | 0.414 | 0.262 | 0.229 | 0.921 | 1.04 | 0.837 | 0.841 |
| | 0.93 | 0.62 | 0.187 | 0.379 | 0.404 | 0.569 | 0.357 | 0.328 | 0.223 | 1.08 | 1.033 | 0.8 | 0.799 |
| | 0.999 | 0.655 | 0.226 | 0.324 | 0.344 | 0.498 | 0.41 | 0.307 | 0.225 | 1.05 | 0.923 | 0.975 | 0.822 |
| | 0.975 | 0.714 | 0.226 | 0.272 | 0.303 | 0.266 | 0.408 | 0.34 | 0.288 | 1.022 | 1.031 | 0.926 | 0.932 |
| | 1.019 | 0.761 | 0.211 | 0.282 | 0.314 | 0.304 | 0.319 | 0.378 | 0.226 | 0.888 | 1.09 | 0.806 | 0.793 |
| | 1.044 | 0.849 | 0.257 | 0.353 | 0.313 | 0.489 | 0.361 | 0.353 | 0.338 | 0.926 | 0.883 | 0.811 | 0.709 |
| Average | 1.0053 | 0.6696 | 0.1984 | 0.3289 | 0.3371 | 0.4327 | 0.3726 | 0.322 | 0.2547 | 1.0041 | 1.0019 | 0.866 | 0.8379 |
| SD | 0.04 | 0.1131 | 0.0464 | 0.0439 | 0.0372 | 0.1098 | 0.0382 | 0.04 | 0.0436 | 0.0946 | 0.0725 | 0.0695 | 0.088 |
| SD/2 | 0.02 | 0.0566 | 0.0232 | 0.022 | 0.0186 | 0.0549 | 0.0191 | 0.02 | 0.0218 | 0.0473 | 0.0362 | 0.0348 | 0.044 |
| % control | 1 | 0.67 | 0.20 | 0.33 | 0.34 | 0.43 | 0.37 | 0.32 | 0.25 | 1.00 | 1.00 | 0.86 | 0.83 |
| H596 | 1.578 | 0.179 | 0.316 | 0.258 | 0.234 | 0.393 | 0.271 | 0.189 | 0.205 | | 1.356 | 1.19 | 1.202 |
| | 1.546 | 0.134 | 0.359 | 0.337 | 0.195 | 0.433 | 0.369 | 0.362 | 0.123 | 1.408 | 1.281 | 1.194 | 1.183 |
| | 1.411 | 0.146 | 0.519 | 0.518 | 0.337 | 0.462 | 0.513 | 0.261 | 0.193 | 1.317 | 1.153 | 1.071 | 1.019 |
| | 1.499 | 0.167 | 0.307 | 0.514 | 0.358 | 0.499 | 0.582 | 0.296 | 0.205 | 1.526 | 1.273 | 1.133 | 1.159 |
| | 1.636 | 0.228 | 0.373 | 0.51 | 0.325 | 0.497 | 0.413 | 0.156 | 0.172 | 1.458 | 1.246 | 1.285 | 1.07 |
| | 1.703 | 0.117 | 0.448 | 0.473 | 0.279 | 0.485 | 0.285 | 0.203 | 0.161 | 1.495 | 1.358 | 1.365 | 0.899 |
| | 1.666 | 0.153 | 0.323 | 0.367 | 0.3 | 0.497 | 0.216 | 0.163 | 0.18 | 1.399 | 1.278 | 1.243 | 0.899 |
| Average | 1.5859 | 0.1606 | 0.3779 | 0.4253 | 0.2897 | 0.4666 | 0.3784 | 0.2329 | 0.177 | 1.4338 | 1.2779 | 1.2116 | 1.0616 |
| SD | 0.0972 | 0.0361 | 0.0786 | 0.1042 | 0.0584 | 0.0404 | 0.1338 | 0.0764 | 0.029 | 0.0753 | 0.0697 | 0.0971 | 0.1283 |
| SD/2 | 0.0486 | 0.018 | 0.0393 | 0.0521 | 0.0292 | 0.0202 | 0.0669 | 0.0382 | 0.0145 | 0.0377 | 0.0349 | 0.0486 | 0.0641 |
| % control | 1 | 0.10 | 0.24 | 0.27 | 0.18 | 0.29 | 0.24 | 0.15 | 0.11 | 0.90 | 0.81 | 0.76 | 0.67 |
| H358 | 0.874 | 0.222 | 0.135 | 0.285 | 0.218 | 0.298 | 0.257 | 0.263 | 0.308 | 0.79 | 0.726 | 0.684 | 0.691 |
| | 0.89 | 0.475 | 0.131 | 0.312 | 0.233 | 0.253 | 0.27 | 0.209 | 0.205 | 0.678 | 0.676 | 0.6 | 0.548 |
| | 0.878 | 0.585 | 0.176 | 0.302 | 0.246 | 0.268 | 0.281 | 0.189 | 0.243 | 0.647 | 0.824 | 0.66 | 0.643 |
| | 0.883 | 0.697 | 0.176 | 0.373 | 0.336 | 0.348 | 0.311 | 0.244 | 0.287 | 0.812 | 0.846 | 0.739 | 0.706 |
| | 0.918 | 0.743 | 0.237 | 0.361 | 0.275 | 0.331 | 0.338 | 0.198 | 0.258 | 0.791 | 0.782 | 0.819 | 0.647 |
| | 0.82 | 0.739 | 0.167 | 0.361 | 0.305 | 0.329 | 0.371 | 0.235 | 0.247 | 0.864 | 0.658 | 0.753 | 0.656 |
| | 0.786 | 0.799 | 0.313 | 0.268 | 0.273 | 0.393 | 0.335 | 0.296 | 0.367 | 0.789 | 0.871 | 0.778 | 0.683 |
| Average | 0.8514 | 0.6086 | 0.1907 | 0.3231 | 0.2694 | 0.3171 | 0.309 | 0.2334 | 0.2736 | 0.7673 | 0.769 | 0.719 | 0.6534 |
| SD | 0.0505 | 0.2029 | 0.0642 | 0.0417 | 0.0414 | 0.0482 | 0.0416 | 0.0381 | 0.0527 | 0.0768 | 0.084 | 0.0752 | 0.0522 |
| SD/2 | 0.0252 | 0.1014 | 0.0321 | 0.0208 | 0.0207 | 0.0241 | 0.0208 | 0.0191 | 0.0264 | 0.0384 | 0.042 | 0.0376 | 0.0261 |
| % control | 1 | 0.71 | 0.22 | 0.38 | 0.32 | 0.37 | 0.36 | 0.27 | 0.32 | 0.90 | 0.90 | 0.84 | 0.77 |
| H460 | 1.652 | 0.044 | 0.254 | 0.452 | 0.322 | 0.092 | 0.143 | 0.185 | 0.071 | 1.531 | 1.473 | 1.297 | 1.191 |
| | 1.706 | 0.091 | 0.225 | 0.369 | 0.283 | 0.412 | 0.107 | 0.159 | 0.467 | 1.338 | 1.427 | 1.323 | 1.216 |
| | 1.675 | 0.164 | 0.273 | 0.249 | 0.25 | 0.24 | 0.138 | 0.143 | 0.127 | 1.635 | 1.518 | 1.388 | 1.047 |
| | 1.727 | 0.226 | 0.326 | 0.257 | 0.353 | 0.191 | 0.161 | 0.1 | 0.144 | 1.354 | 1.504 | 1.304 | 1.094 |
| | 1.633 | 0.258 | 0.254 | 0.235 | 0.352 | 0.228 | 0.242 | 0.104 | 0.138 | 1.477 | 1.524 | 1.362 | 0.946 |
| | 1.671 | 0.263 | 0.321 | 0.193 | 0.408 | 0.242 | 0.268 | 0.117 | 0.118 | 1.476 | 1.575 | 1.335 | 1.108 |
| | 1.84 | 0.262 | 0.377 | 0.142 | 0.32 | 0.335 | 0.177 | 0.241 | 0.169 | 1.534 | 1.534 | 1.307 | 1.135 |
| Average | 1.7168 | 0.1869 | 0.29 | 0.271 | 0.3269 | 0.2486 | 0.1766 | 0.1499 | 0.1763 | 1.4779 | 1.5079 | 1.3309 | 1.1053 |
| SD | 0.068 | 0.0896 | 0.0531 | 0.1057 | 0.0513 | 0.1021 | 0.0582 | 0.0506 | 0.1316 | 0.1045 | 0.0471 | 0.0335 | 0.0907 |
| SD/2 | 0.034 | 0.0448 | 0.0266 | 0.0529 | 0.0257 | 0.0511 | 0.0291 | 0.0253 | 0.0658 | 0.0523 | 0.0236 | 0.0168 | 0.0454 |
| % control | 1 | 0.11 | 0.17 | 0.16 | 0.19 | 0.14 | 0.10 | 0.09 | 0.10 | 0.86 | 0.88 | 0.78 | 0.64 |
| | | 12-Hydroxyrotenone | | | | 12,12a-dehydrorotenone | | | | Isorotenone | | | |
| | | (Drug 7) | | | | (Drug 8) | | | | (Drug 9) | | | |
| | Control | 0.01 μM | 0.1 μM | 0.5 μM | 1 μM | 0.01 μM | 0.1 μM | 0.5 μM | 1 μM | 0.01 μM | 0.1 μM | 0.5 μM | 1 μM |
| A549 | 0.703 | 0.485 | 0.335 | 0.116 | 0.186 | 0.782 | 0.509 | 0.333 | 0.603 | 0.612 | 0.195 | 0.195 | 0.201 |
| | 0.684 | 0.477 | 0.42 | 0.146 | 0.141 | 0.836 | 0.555 | 0.429 | 0.465 | 0.449 | 0.181 | 0.285 | 0.198 |
| | 0.974 | 0.379 | 0.383 | 0.212 | 0.181 | 0.926 | 0.521 | 0.45 | 0.472 | 0.603 | 0.205 | 0.336 | 0.237 |
| | 0.723 | 0.795 | 0.416 | 0.258 | 0.201 | 0.753 | 0.689 | 0.556 | 0.603 | 0.511 | 0.182 | 0.395 | 0.199 |
| | 0.738 | 0.452 | 0.399 | 0.174 | 0.19 | 0.798 | 0.729 | 0.463 | 0.487 | 0.716 | 0.263 | 0.413 | 0.142 |
| | 0.824 | 0.535 | 0.443 | 0.237 | 0.208 | 0.803 | 0.762 | 0.634 | 0.486 | 0.769 | 0.269 | 0.443 | 0.132 |
| | 0.765 | 0.509 | 0.505 | 0.211 | 0.194 | 0.671 | 0.731 | 0.643 | 0.52 | 0.577 | 0.281 | 0.478 | 0.15 |
| Average | 0.78625 | 0.51886 | 0.41443 | 0.19343 | 0.18586 | 0.79557 | 0.64229 | 0.50114 | 0.51943 | 0.60529 | 0.22514 | 0.36357 | 0.17986 |
| SD | 0.0996 | 0.13137 | 0.05258 | 0.05057 | 0.02175 | 0.0777 | 0.10954 | 0.11426 | 0.05965 | 0.11047 | 0.04397 | 0.09847 | 0.03878 |
| SD/2 | 0.0498 | 0.06568 | 0.02629 | 0.02528 | 0.01088 | 0.03885 | 0.05477 | 0.05713 | 0.02983 | 0.05524 | 0.02199 | 0.04924 | 0.01939 |
| % control | 1 | 0.66 | 0.53 | 0.25 | 0.24 | 1.01 | 0.82 | 0.64 | 0.66 | 0.77 | 0.29 | 0.46 | 0.23 |
| H596 | 1.145 | 0.588 | 0.569 | 0.182 | 0.397 | 0.914 | 1.279 | 0.538 | 0.691 | 1.053 | 0.74 | 0.163 | 0.213 |
| | 1.695 | 0.455 | 0.75 | 0.27 | 0.319 | 1.381 | 1.115 | 0.428 | 0.674 | 1.355 | 0.467 | 0.196 | 0.254 |
| | 1.782 | 0.687 | 0.627 | 0.239 | 0.308 | 1.053 | 1.303 | 0.51 | 0.502 | 1.251 | 0.698 | 0.171 | 0.181 |
| | 1.7 | 0.783 | 0.653 | 0.202 | 0.399 | 1.104 | 1.005 | 0.669 | 0.658 | 1.142 | 0.542 | 0.234 | 0.206 |
| | 1.819 | 0.747 | 0.673 | 0.195 | 0.421 | 1.204 | 1.128 | 0.597 | 0.722 | 1.156 | 0.507 | 0.234 | 0.215 |
| | 1.599 | 0.761 | 0.614 | 0.21 | 0.234 | 1.345 | 0.862 | 0.342 | 0.632 | 1.041 | 0.476 | 0.271 | 0.187 |
| | 1.681 | 0.84 | 0.593 | 0.215 | 0.244 | 1.295 | 1.221 | 0.756 | 0.485 | 1.205 | 0.58 | 0.266 | 0.261 |
| Average | 1.591 | 0.69443 | 0.63986 | 0.21614 | 0.33171 | 1.18514 | 1.13043 | 0.54857 | 0.62343 | 1.17186 | 0.57286 | 0.21929 | 0.21671 |
| SD | 0.23699 | 0.13225 | 0.0598 | 0.02965 | 0.07608 | 0.17025 | 0.1571 | 0.14064 | 0.09311 | 0.11053 | 0.10765 | 0.04346 | 0.03066 |
| SD/2 | 0.11849 | 0.06612 | 0.0299 | 0.01483 | 0.03804 | 0.08512 | 0.07855 | 0.07032 | 0.04655 | 0.05526 | 0.05382 | 0.02173 | 0.01533 |
| % control | 1 | 0.44 | 0.40 | 0.14 | 0.21 | 0.74 | 0.71 | 0.34 | 0.39 | 0.74 | 0.36 | 0.14 | 0.14 |
| H460 | 1.416 | 0.493 | 0.519 | 0.177 | 0.185 | 1.101 | 0.957 | 0.395 | 0.379 | 1.088 | 0.381 | 0.283 | 0.259 |
| | 1.437 | 0.554 | 0.501 | 0.241 | 0.196 | 1.267 | 1.171 | 0.425 | 0.309 | 1.1 | 0.425 | 0.324 | 0.277 |
| | 1.43 | 0.65 | 0.443 | 0.209 | 0.153 | 1.233 | 0.981 | 0.316 | 0.429 | 1.105 | 0.49 | 0.28 | 0.212 |
| | 1.324 | 0.711 | 0.398 | 0.225 | 0.136 | 1.22 | 1.039 | 0.396 | 0.436 | 1.087 | 0.386 | 0.32 | 0.271 |
| | 1.439 | 0.718 | 0.513 | 0.268 | 0.301 | 1.26 | 0.919 | 0.422 | 0.4 | 1.101 | 0.404 | 0.288 | 0.179 |
| | 1.566 | 0.708 | 0.577 | 0.334 | 0.181 | 1.238 | 1.069 | 0.47 | 0.504 | 1.117 | 0.399 | 0.253 | 0.23 |
| | 1.508 | 0.786 | 0.585 | 0.292 | 0.177 | 1.529 | 0.987 | 0.549 | 0.412 | 1.216 | 0.305 | 0.204 | 0.164 |
| Average | 1.48089 | 0.66 | 0.50514 | 0.24943 | 0.18986 | 1.264 | 1.01757 | 0.42471 | 0.40986 | 1.11629 | 0.39857 | 0.27886 | 0.22743 |
| SD | 0.09659 | 0.10277 | 0.06727 | 0.05301 | 0.0531 | 0.12935 | 0.08397 | 0.07192 | 0.05936 | 0.04514 | 0.05524 | 0.04104 | 0.04466 |
| SD/2 | 0.04829 | 0.05138 | 0.03364 | 0.0265 | 0.02655 | 0.06468 | 0.04198 | 0.03596 | 0.02968 | 0.02257 | 0.02762 | 0.02052 | 0.02233 |
| % control | 1 | 0.45 | 0.34 | 0.17 | 0.13 | 0.85 | 0.69 | 0.29 | 0.28 | 0.75 | 0.27 | 0.19 | 0.15 |
| H1299 | 0.986 | 0.369 | 0.478 | 0.143 | 0.158 | 0.495 | 0.614 | 0.373 | 0.383 | 0.596 | 0.29 | 0.211 | 0.335 |
| | 0.779 | 0.435 | 0.547 | 0.173 | 0.166 | 0.744 | 0.603 | 0.429 | 0.453 | 0.571 | 0.342 | 0.282 | 0.431 |
| | 0.867 | 0.468 | 0.434 | 0.238 | 0.254 | 0.766 | 0.689 | 0.439 | 0.303 | 0.617 | 0.199 | 0.281 | 0.238 |
| | 0.871 | 0.548 | 0.264 | 0.221 | 0.237 | 0.824 | 0.699 | 0.284 | 0.492 | 0.69 | 0.492 | 0.392 | 0.228 |
| | 0.871 | 0.57 | 0.495 | 0.106 | 0.205 | 0.832 | 0.528 | 0.371 | 0.534 | 0.55 | 0.553 | 0.341 | 0.244 |
| | 0.797 | 0.611 | 0.557 | 0.166 | 0.216 | 0.853 | 0.681 | 0.344 | 0.496 | 0.604 | 0.607 | 0.358 | 0.198 |
| | 0.725 | 0.566 | 0.478 | 0.169 | 0.198 | 0.818 | 0.608 | 0.484 | 0.365 | 0.568 | 0.664 | 0.174 | 0.183 |
| | 0.943 | | | | | | | | | | | | |
| | 0.859 | | | | | | | | | | | | |
| Average | 0.85533 | 0.50957 | 0.46471 | 0.17371 | 0.20486 | 0.76171 | 0.63171 | 0.38914 | 0.43229 | 0.59943 | 0.44957 | 0.29129 | 0.26529 |
| SD | 0.08032 | 0.08723 | 0.09814 | 0.04468 | 0.03494 | 0.12369 | 0.0615 | 0.06678 | 0.08374 | 0.04616 | 0.17468 | 0.07901 | 0.08776 |
| SD/2 | 0.4016 | 0.04361 | 0.04907 | 0.02234 | 0.01747 | 0.06184 | 0.03075 | 0.03339 | 0.04187 | 0.02308 | 0.08734 | 0.03951 | 0.04388 |
| % control | 1 | 0.60 | 0.54 | 0.20 | 0.24 | 0.89 | 0.74 | 0.45 | 0.51 | 0.70 | 0.53 | 0.34 | 0.31 |
| | | 4-chlororot-2′-enoic acid | | | | 1,2-dihydrodeguelin | | | | 2-phenylselenyl-1,2-dihydrodeguelin | | | |
| | | (Drug 10) | | | | (Drug 11) | | | | (Drug 12) | | | |
| | Control | 0.01 μM | 0.1 μM | 0.5 μM | 1 μM | 0.01 μM | 0.1 μM | 0.5 μM | 1 μM | 0.01 μM | 0.1 μM | 0.5 μM | 1 μM |
| A549 | 0.637 | 0.285 | 0.326 | 0.252 | 0.28 | 0.779 | 0.343 | 0.355 | 0.303 | 0.58 | 0.604 | 0.533 | 0.534 |
| | 0.56 | 0.364 | 0.312 | 0.237 | 0.293 | 0.766 | 0.313 | 0.444 | 0.302 | 0.633 | 0.545 | 0.614 | 0.536 |
| | 0.544 | 0.412 | 0.326 | 0.276 | 0.327 | 0.723 | 0.358 | 0.514 | 0.313 | 0.616 | 0.597 | 0.532 | 0.557 |
| | 0.578 | 0.407 | 0.337 | 0.273 | 0.294 | 0.736 | 0.362 | 0.418 | 0.359 | 0.636 | 0.519 | 0.584 | 0.571 |
| | 0.554 | 0.434 | 0.363 | 0.286 | 0.282 | 0.77 | 0.405 | 0.47 | 0.422 | 0.715 | 0.606 | 0.567 | 0.586 |
| | 0.562 | 0.411 | 0.341 | 0.305 | 0.343 | 0.75 | 0.375 | 0.413 | 0.316 | 0.577 | 0.719 | 0.553 | 0.586 |
| | 0.568 | 0.446 | 0.378 | 0.281 | 0.285 | 0.695 | 0.435 | 0.398 | 0.4 | 0.601 | 0.713 | 0.585 | 0.621 |
| Average | 0.57744 | 0.39414 | 0.34043 | 0.27286 | 0.30057 | 0.74557 | 0.37014 | 0.43029 | 0.345 | 0.62257 | 0.61471 | 0.56686 | 0.57014 |
| SD | 0.03446 | 0.05457 | 0.02294 | 0.02237 | 0.02453 | 0.02975 | 0.04013 | 0.05153 | 0.04938 | 0.04696 | 0.07644 | 0.03 | 0.03092 |
| SD/2 | 0.01723 | 0.02728 | 0.01147 | 0.01119 | 0.01226 | 0.01487 | 0.02006 | 0.02577 | 0.02469 | 0.02348 | 0.03822 | 0.015 | 0.01546 |
| % control | 1 | 0.68 | 0.59 | 0.47 | 0.52 | 1.29 | 0.64 | 0.75 | 0.60 | 1.08 | 1.06 | 0.98 | 0.99 |
| H596 | 1.559 | 0.549 | 0.399 | 0.351 | 0.238 | 1.286 | 0.677 | 0.469 | 0.385 | 1.468 | 1.911 | 1.686 | 1.143 |
| | 1.656 | 0.717 | 0.425 | 0.385 | 0.275 | 1.422 | 0.894 | 0.448 | 0.377 | 1.111 | 1.663 | 1.82 | 1.448 |
| | 1.763 | 0.91 | 0.317 | 0.428 | 0.287 | 1.376 | 0.729 | 0.439 | 0.447 | 1.465 | 1.92 | 1.177 | 1.604 |
| | 1.647 | 0.825 | 0.319 | 0.472 | 0.343 | 1.041 | 0.59 | 0.322 | 0.585 | 1.374 | 1.666 | 1.899 | 1.637 |
| | 1.73 | 0.869 | 0.432 | 0.388 | 0.315 | 1.247 | 0.476 | 0.282 | 0.404 | 1.701 | 1.398 | 1.872 | 1.497 |
| | 1.545 | 0.988 | 0.374 | 0.424 | 0.3 | 1.293 | 0.579 | 0.424 | 0.429 | 1.481 | 1.603 | 2.048 | 1.557 |
| | 1.787 | 0.854 | 0.448 | 0.402 | 0.443 | 1.168 | 0.596 | 0.508 | 0.445 | 1.344 | 1.623 | 1.655 | 1.828 |
| Average | 1.661 | 0.816 | 0.38771 | 0.40714 | 0.31443 | 1.26186 | 0.64871 | 0.41314 | 0.43886 | 1.42057 | 1.68343 | 1.73671 | 1.53057 |
| SD | 0.09159 | 0.14367 | 0.05327 | 0.03865 | 0.06545 | 0.12782 | 0.13437 | 0.08127 | 0.07012 | 0.17813 | 0.1826 | 0.28018 | 0.20978 |
| SD/2 | 0.0458 | 0.07183 | 0.02663 | 0.01932 | 0.03273 | 0.06391 | 0.06718 | 0.04063 | 0.03506 | 0.08906 | 0.0913 | 0.14009 | 0.10489 |
| % control | 1 | 0.49 | 0.23 | 0.25 | 0.19 | 0.76 | 0.39 | 0.25 | 0.26 | 0.86 | 1.01 | 1.05 | 0.92 |
| H460 | 1.222 | 0.325 | 0.334 | 0.397 | 0.226 | 0.67 | 0.352 | 0.372 | 0.314 | 0.763 | 0.603 | 0.621 | 0.709 |
| | 1.234 | 0.307 | 0.376 | 0.335 | 0.237 | 0.672 | 0.354 | 0.35 | 0.376 | 0.753 | 0.593 | 0.714 | 0.659 |
| | 1.222 | 0.376 | 0.376 | 0.481 | 0.241 | 0.578 | 0.375 | 0.36 | 0.344 | 0.691 | 0.691 | 0.653 | 0.698 |
| | 1.073 | 0.46 | 0.331 | 0.39 | 0.254 | 0.697 | 0.234 | 0.296 | 0.314 | 0.648 | 0.781 | 0.739 | 0.79 |
| | 1.133 | 0.538 | 0.351 | 0.373 | 0.3 | 0.719 | 0.26 | 0.2577 | 0.302 | 0.765 | 0.732 | 0.77 | 0.717 |
| | 1.111 | 0.472 | 0.364 | 0.322 | 0.316 | 0.651 | 0.303 | 0.283 | 0.291 | 0.774 | 0.815 | 0.758 | 0.805 |
| | 1.204 | 0.58 | 0.446 | 0.427 | 0.304 | 0.678 | 0.372 | 0.432 | 0.366 | 0.822 | 0.832 | 0.734 | 0.806 |
| Average | 1.17156 | 0.43686 | 0.36829 | 0.38929 | 0.26829 | 0.66643 | 0.32143 | 0.33581 | 0.32957 | 0.74514 | 0.721 | 0.71271 | 0.74057 |
| SD | 0.05634 | 0.10454 | 0.03882 | 0.05421 | 0.03713 | 0.0446 | 0.05654 | 0.06031 | 0.03272 | 0.05757 | 0.09668 | 0.05546 | 0.05901 |
| SD/2 | 0.02817 | 0.05227 | 0.01941 | 0.02711 | 0.01857 | 0.0223 | 0.02827 | 0.03015 | 0.01636 | 0.02878 | 0.04834 | 0.02773 | 0.02951 |
| % control | 1 | 0.37 | 0.31 | 0.33 | 0.23 | 0.57 | 0.27 | 0.29 | 0.28 | 0.64 | 0.62 | 0.61 | 0.63 |
| H1299 | 0.899 | 0.284 | 0.457 | 0.272 | 0.242 | 0.573 | 0.302 | 0.275 | 0.157 | 0.513 | 0.514 | 0.637 | 0.434 |
| | 0.812 | 0.595 | 0.393 | 0.398 | 0.264 | 0.553 | 0.338 | 0.579 | 0.202 | 0.438 | 0.55 | 0.549 | 0.41 |
| | 0.863 | 0.461 | 0.448 | 0.424 | 0.221 | 0.567 | 0.317 | 0.324 | 0.236 | 0.626 | 0.568 | 0.636 | 0.413 |
| | 0.749 | 0.392 | 0.421 | 0.319 | 0.26 | 0.483 | 0.271 | 0.32 | 0.233 | 0.426 | 0.409 | 0.614 | 0.451 |
| | 0.713 | 0.452 | 0.496 | 0.365 | 0.308 | 0.493 | 0.215 | 0.398 | 0.217 | 0.609 | 0.465 | 0.847 | 0.378 |
| | 0.708 | 0.503 | 0.385 | 0.389 | 0.357 | 0.424 | 0.272 | 0.294 | 0.262 | 0.729 | 0.466 | 0.655 | 0.357 |
| | 0.734 | 0.475 | 0.415 | 0.315 | 0.294 | 0.42 | 0.278 | 0.369 | 0.218 | 0.515 | 0.527 | 0.458 | 0.358 |
| Average | 0.768 | 0.45171 | 0.43071 | 0.35457 | 0.278 | 0.50186 | 0.28471 | 0.36557 | 0.21786 | 0.55086 | 0.49986 | 0.628 | 0.40014 |
| SD | 0.07201 | 0.09616 | 0.03896 | 0.05424 | 0.04557 | 0.0647 | 0.03966 | 0.10307 | 0.03282 | 0.1094 | 0.05584 | 0.11844 | 0.03679 |
| SD/2 | 0.03601 | 0.04808 | 0.01948 | 0.02712 | 0.02279 | 0.03235 | 0.01983 | 0.05154 | 0.01641 | 0.0547 | 0.02792 | 0.05922 | 0.0184 |
| % control | 1 | 0.59 | 0.56 | 0.46 | 0.36 | 0.65 | 0.37 | 0.48 | 0.28 | 0.72 | 0.65 | 0.82 | 0.52 |

Bromorot-2′-enoic acid (Drug 13)

| | Control | 0.01 μM | 0.1 μM | 0.5 μM | 1 μM |
|---|---|---|---|---|---|
| A549 | 1.793 | 0.59 | 0.551 | 0.151 | 0.236 |
| | 1.677 | 0.64 | 0.561 | 0.175 | 0.182 |
| | 1.628 | 0.629 | 0.629 | 0.249 | 0.208 |
| | 1.714 | 0.686 | 0.683 | 0.285 | 0.231 |
| | 1.728 | 0.772 | 0.728 | 0.383 | 0.253 |
| | 1.801 | 0.822 | 0.732 | 0.424 | 0.303 |
| | 1.793 | 0.811 | 0.763 | 0.407 | 0.256 |
| Average | 1.73343 | 0.70714 | 0.66386 | 0.29629 | 0.23843 |
| SD | 0.06629 | 0.09396 | 0.08513 | 0.11126 | 0.03843 |
| SD/2 | 0.03315 | 0.04698 | 0.04257 | 0.05563 | 0.01922 |
| % control | 1 | 0.41 | 0.38 | 0.17 | 0.14 |
| H596 | 1.913 | 0.165 | 0.911 | 0.151 | 0.093 |
| | 1.899 | 0.226 | 0.885 | 0.113 | 0.249 |
| | 1.943 | 0.172 | 0.902 | 0.154 | 0.262 |
| | 2 | 0.217 | 0.912 | 0.166 | 0.239 |
| | 1.899 | 1.071 | 0.914 | 0.181 | 0.269 |
| | 1.908 | 0.332 | 0.865 | 0.193 | 0.241 |
| | 2.001 | 0.283 | 0.96 | 0.239 | 0.238 |
| Average | 1.93757 | 0.35229 | 0.907 | 0.171 | 0.22729 |
| SD | 0.04547 | 0.32238 | 0.02936 | 0.03934 | 0.0604 |
| SD/2 | 0.02273 | 0.16119 | 0.01468 | 0.01967 | 0.0302 |
| % control | 1 | 0.18 | 0.47 | 0.09 | 0.12 |
| H460 | 2.071 | 0.649 | 0.368 | 0.185 | 0.168 |
| | 2.121 | 0.591 | 0.37 | 0.214 | 0.201 |
| | 2.086 | 0.663 | 0.441 | 0.251 | 0.237 |
| | 2.113 | 0.771 | 0.443 | 0.319 | 0.224 |
| | 2.11 | 0.863 | 0.456 | 0.323 | 0.289 |
| | 2.114 | 0.737 | 0.557 | 0.37 | 0.269 |
| | 2.249 | 0.808 | 0.599 | 0.406 | 0.338 |
| Average | 2.12343 | 0.726 | 0.462 | 0.29543 | 0.24657 |
| SD | 0.05817 | 0.0964 | 0.08749 | 0.08155 | 0.05703 |
| SD/2 | 0.02908 | 0.0482 | 0.04375 | 0.04077 | 0.02851 |
| % control | 1 | 0.34 | 0.22 | 0.14 | 0.12 |
| H1299 | 1.386 | 0.44 | 0.27 | 0.181 | 0.216 |
| | 1.387 | 0.49 | 0.226 | 0.185 | 0.234 |
| | 1.379 | 0.511 | 0.271 | 0.216 | 0.288 |
| | 1.308 | 0.562 | 0.289 | 0.241 | 0.316 |
| | 1.299 | 0.563 | 0.303 | 0.252 | 0.296 |
| | 1.418 | 0.617 | 0.345 | 0.245 | 0.314 |
| | 1.518 | 0.572 | 0.393 | 0.336 | 0.28 |
| Average | 1.385 | 0.53643 | 0.29957 | 0.23657 | 0.27771 |
| SD | 0.07319 | 0.05947 | 0.0548 | 0.05226 | 0.03862 |
| SD/2 | 0.0366 | 0.02973 | 0.0274 | 0.02613 | 0.01931 |
| % control | 1 | 0.39 | 0.22 | 0.17 | 0.20 |
| H322 | 0.44 | 0.214 | 0.173 | 0.15 | 0.219 |
| | 0.492 | 0.206 | 0.18 | 0.247 | 0.301 |
| | 0.449 | 0.287 | 0.195 | 0.257 | 0.345 |
| | 0.522 | 0.292 | 0.239 | 0.225 | 0.513 |
| | 0.549 | 0.347 | 0.25 | 0.323 | 0.449 |
| | 0.449 | 0.404 | 0.294 | 0.345 | 0.504 |
| | 0.504 | 0.37 | 0.363 | 0.387 | 0.493 |
| Average | 0.48643 | 0.30286 | 0.242 | 0.27629 | 0.40343 |
| SD | 0.04179 | 0.07564 | 0.0686 | 0.08059 | 0.11557 |
| SD/2 | 0.02089 | 0.03782 | 0.0343 | 0.0403 | 0.05779 |
| % control | 1 | 0.62 | 0.50 | 0.57 | 0.83 |
| H358 | | 0.246 | 0.163 | 0.214 | 0.212 |
| | 0.549 | 0.237 | 0.207 | 0.218 | 0.1 |
| | 0.547 | 0.24 | 0.208 | 0.218 | 0.198 |
| | 0.575 | 0.304 | 0.235 | 0.221 | 0.361 |
| | 0.582 | 0.315 | 0.257 | 0.313 | 0.219 |
| | 0.647 | 0.351 | 0.249 | 0.296 | 0.351 |
| | 0.617 | 0.298 | 0.307 | 0.3 | 0.299 |
| Average | 0.59657 | 0.28443 | 0.23229 | 0.25429 | 0.24857 |
| SD | 0.0452 | 0.04403 | 0.04566 | 0.0459 | 0.09356 |
| SD/2 | 0.0226 | 0.02202 | 0.02283 | 0.02295 | 0.04678 |
| % control | 1 | 0.48 | 0.39 | 0.43 | 0.42 |

### Example 7

### Antitumor and Anti-Angiogenic Activity of Deguelin

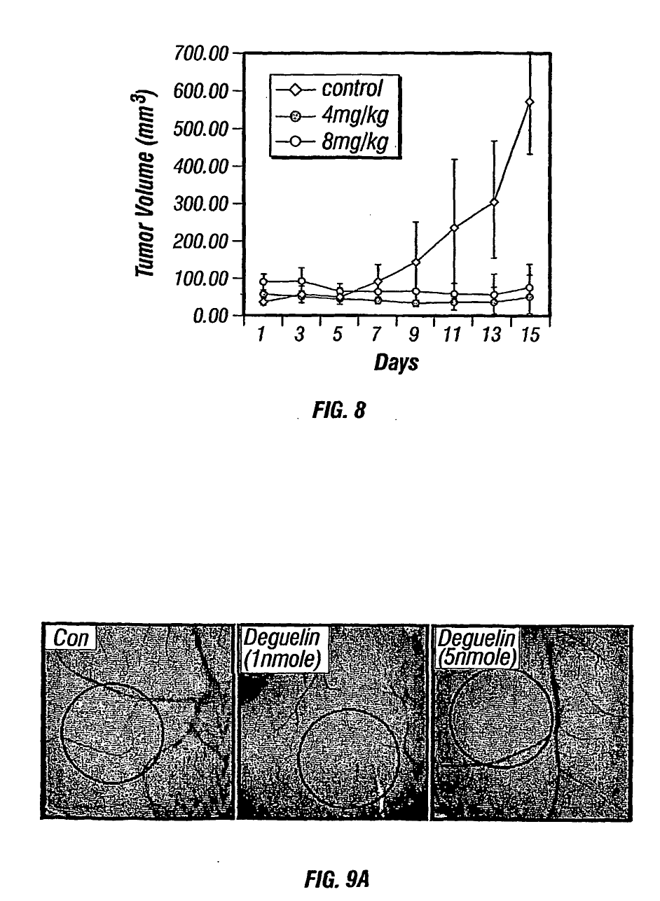

The antitumor effect of deguelin was tested using an iii vivo model. H1299 cells were injected into the dorsal flank of athymic nude mice. Once tumor volume reached 40-80 mm³, treatment for 5 consecutive days with 4 or 8 mg/kg deguelin began. Tumors were measured every other day for 15 days. Growth of NSCLC xenografts was found to be inhibited by treatment of deguelin at 4 mg/kg or 8 mg/kg concentrations compared to the control (FIG. 8). The results are expressed as the mean (±SD) tumor volume (calculated from at least 5 mice) relative to the initial volume.

The anti-angiogenic activity of deguelin was assessed using a CAM assay. Chick embryos were incubation for 3 days after which, about 2 ml of egg albumin was removed from the embryo with a hypodermic needle to allow the CAM and yolk sac to drop away from the shell membrane. On day 3.5, the shell was punched out and removed and the shell membrane was peeled away. For testing of inhibition of angiogenesis, sample-loaded Thermanox™ coverslips containing vehicle control, 1-5 μM of deguelin, or 1 μg of retinoic acid (RA) as a positive control were air dried and applied to the chorioallantoic membrane (CAM) surface of 4.5-day-old chick embryos, and the embryos were incubated for 2 days. After the two day incubation period, 500 μl of 10% fat emulsion was injected into the chorioallantoic membrane and observed microscopically.

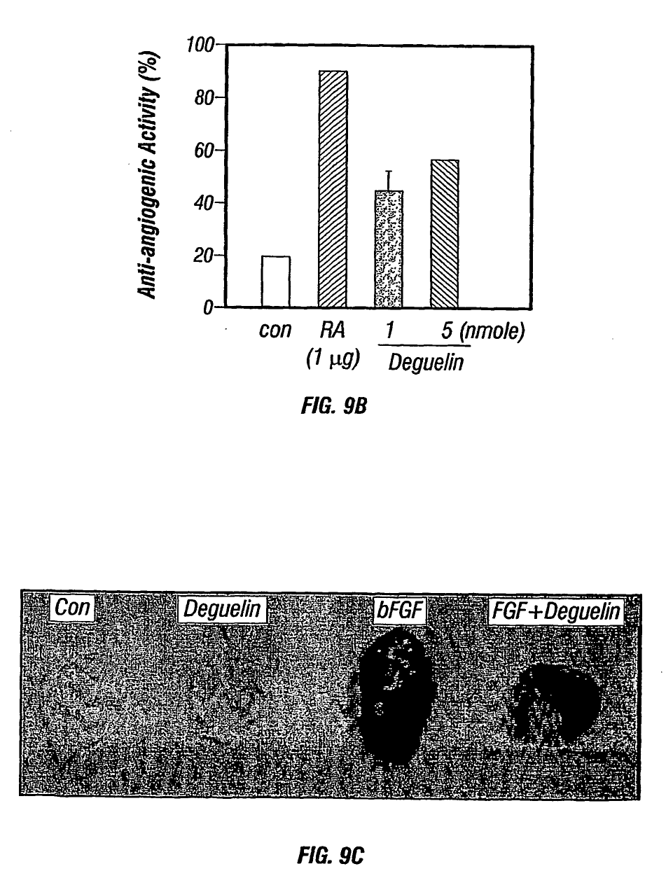

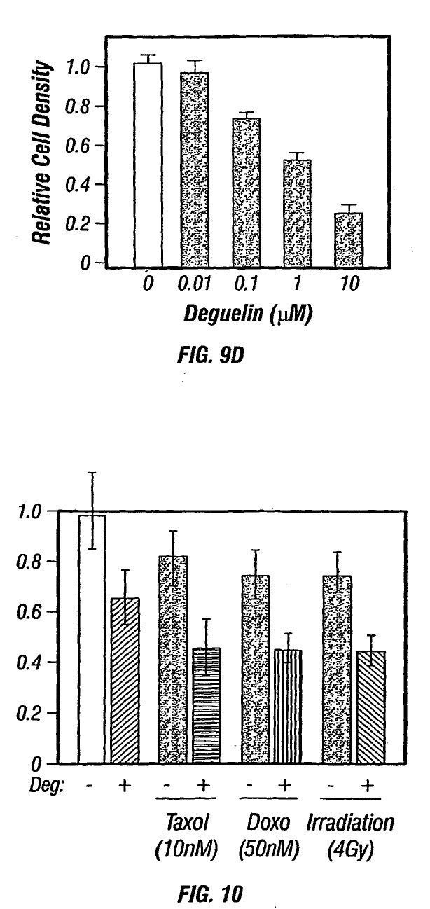

Retinoic acid, an anti-angiogenic compound, was used as a positive control for determining anti-angiogenic response. When the deguelin-treated CAM showed an a vascular zone to a degree similar to that of the the RA-treated CAM, the response was scored as positive, and results were calculated as the percentage of positive eggs among the total number of eggs tested. This independent experiment was repeated three times with more than 20 eggs. Treatment with deguelin substantially reduced new vessel formation in chick embryos without any signs of thrombosis and hemorrhage and with negligible egg lethality (FIG. 9A; circle indicate the placement of coverslip. The anti-angiogenic activity of deguelin (5 nmol/egg) was 59.2% (FIG. 9B.).

Inhibition of angiogenesis by deguelin was also evaluated using the Matrigel™ plug assay, an established in vivo angiogenesis model, using nude mice. Matrigel alone was used as a negative control, 100 ng/ml bFGF and 72 units/ml heparin in a vehicle of 0.1% BSA/PBS (bFGF) for a positive control, 5 nmole deguelin alone, and 5 nmole deguelin plus bFGF were included. Seven days after injection, mice were sacrificed. Control plugs, in which Matrigel™ was injected with heparin alone, showed few vessels, but basic fibroblast growth factor (bFGF) at 100 ng/ml) strongly enhanced vessel development in the plugs. Deguelin was found to markedly inhibit bFGF-induced angiogenesis (FIG. 9C). Moreover, deguelin effectively suppressed proliferation of HUVEC cells treated with 0.01, 0.1, 1 or 10 μM of deguelin for 3 days (FIG. 9D). Inhibition of cell proliferation was observed at 0. 1, 1 and 10 μM concentrations of deguelin in these cells. All of these findings indicated the anti-angiogenic activity of deguelin.

### Example 8

### Deguelin Enhances the Efficacy of Chemotherapeutic Agents

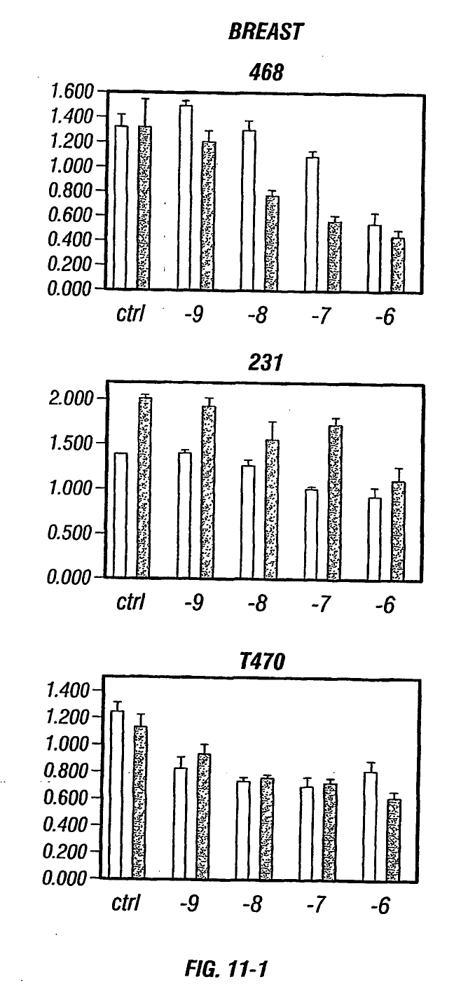

Additional studies were conducted to determine whether deguelin can sensitize cells resistant to a chemotherapeutic agent. H1299 NSCLC cells were incubated with deguelin (100 nM) for 2 days. 10 nM paclitaxel (taxol), 50 nM doxorubicin, or 4Gy of irradiation (Rad) were added for 1 day of deguelin treatment followed by MTT analysis (FIG. 10). The results showed that deguelin sensitizes cancer cells to chemotherapeutic agents and enhances the growth inhibitory effect of these agents.

### Example 9

### Deguelin Inhibits Cell Growth Proliferation in other Cancer Cells

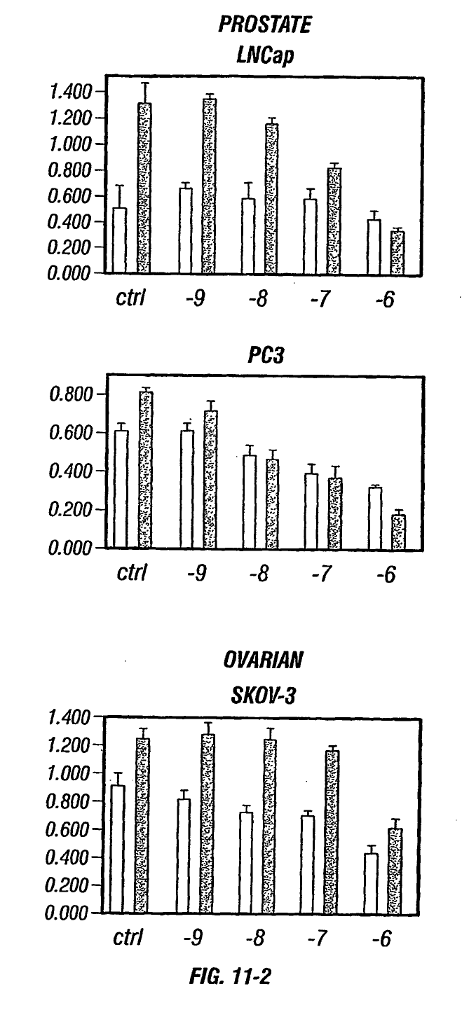

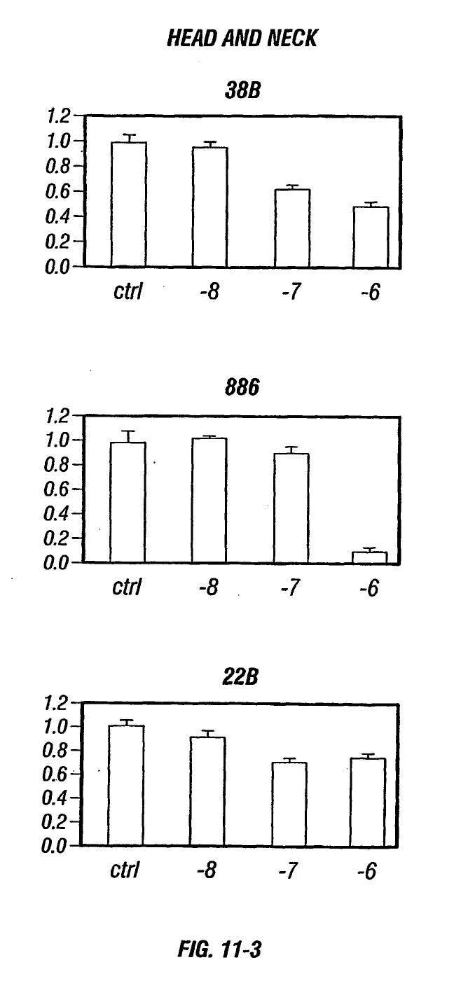

It was next determined whether deguelin can inhibit cell growth in cancer cells other than lung cancer cells. Cancer cells, such as breast, prostate, head & neck and ovarian cells were treated with 10⁻⁶ M, 10⁻⁷ M, 10⁻⁸ M, or 10⁻⁹ M deguelin for 3 or 5 days, and cell growth inhibition analyzed by MTT assay. Deguelin was found to inhibit cell proliferation in these cells in a dose dependent manner. Cell proliferation was found to more effectively inhibited at a concentration of 10⁻⁷ M to 10⁻⁶ M (FIG. 11). This data thus, supports the results observed in lung cancer cells and provides deguelin is an effective chemopreventive agent in treating various cancers.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,690,915
U.S. Pat. No. 5,199,942
Ahmadian et al.i, Proc. Natl. Acad. Sci. USA, 96:7065-7070, 1999.
Anzenveno, J. Org. Chem., 44:2578-2580, 1979.
Arap et al., Cancer Res., 55(6):1351-1354, 1995.
Bakhshi et al., Cell, 41(3):899-906, 1985.
Benner et al., Cancer Res., 52:2758S-2763S, 1992.
Boulton and Cobb, Cell Regul., 2:357, 1991.
Brognard et al., Cancer Res., 61:3986-3997, 2001.
Caldas et al., Nat. Genet., 8(1):27-32, 1994.
California Dept. Fish and Game, In: Lake Davis Northern Pike Eradication Project, 1997.
Cantley et al., Proc. Natl. Acad. Sci. USA, 96:4240-4255, 1999.
Chang et al., Science, 276_{—}1848-1850, 1997.
Cheng et al., Cancer Res., 54(21):5547-5551, 1994.
Cleary and Sklar, Proc. Natl. Acad. Sci. USA, (21):7439-7443, 1985.
Cleary et al., J. Exp. Med., 164(1):315-320, 1986.
Cunningham et al., Cancer Lett., 95:93-97, 1995.
Di Cristofano et al., Cell, 100:387-390, 2000.
Favata, J. Biol. Chem., 273:18623, 1998.
Figueras and Gosalvez, Eur. J Cancer, 9:529-531, 1973.
Franke et al., Cell, 81:727-736, 1995.
Fukami and Wilkinson, In: Insecticide Biochemistry and Physiology, Plenum, N.Y., 1976.
Fukami et al., In: Naturally Occurring Insecticides, Dekker, N.Y., 1971.
Gerhäuser et al., Am. Chem. Soc. Abstr., 211(1-2):81, 1996.
Gerhauser et al., Nat. Med., 1:260-266, 1995.
Gosalvez et al., Cancer Treat. Rep., 60:1-8, 1976.
Grimberg et al., J. Cell Physiol., 183:1-9, 2000.
Hansen et al., Toxicol. Appl. Pharmacol., 7:535-542, 1965.
Hollingworth and Ahammadsahib, Rev. Pestic. Toxicol., 3:277-302, 1995.
Hollstein et al., Science, 253(5015):49-53, 1991.
Hong et al., Science, 278:1073-1077, 1997.
Huang and Ferrell, Proc. Natl. Acad. Sci. USA, 93:10078, 1996.
Hussussian et at., Nat. Genet., 8(1):15-21, 1994.
Jang et al., Science, 275:218-220, 1997.
Jetten et al., Biochem. Soc. Trans., 14:930-933, 1986.
Ji et al., Cancer Res., 62:2715-2720, 2002.
Jimenez et al., EMBO J, 17:743-753, 1998.
Kalb et al., J. Biol. Chem., 271:28097, 1996.
Kamb et al., Nat. Genet., 8(1):23-2, 1994.
Kelloff et al., Cancer Res., 54:2015S-2024S, 1994.
Kerr et al, Br. J Cancer, 26(4):239-257, 1972.
Khuri et al., Annals of Oncology, 12:739-744,2001.
Kim et al., Cancer Res., 55:5603-5610, 1995.
Kinzler et al., Cell, 97:159-150, 1996.
Klein-Szanto et al., Proc. Natl. Acad. Sci. usa, 89:6693-6697, 1992.
Klippel et al., Mol. Cell Biol., 18:5699-5711, 1998.
Kobayashi et al., Proc. Natl. Acad. Sci. USA, 96:4874-4879, 1999.
Lee et al., Cancer Res., 62(12):3530-3537, 2002.
Lee et al, Cell Groth Differ., 7:997-1004, 1996.
Lee et al., J. Natl. Cancer Instit., 93(14):1081-1088, 2001.
Lewis et al., Adv. Cancer Res., 74:79, 1998.
Li et al., J. Virol., 72(3):2055-2061, 1998.
Lotan, FASEB J., 10:1031-1039, 1996.
Luyengi et al., Phytochem., 36:1523-1526, 1994.
Maehama et al., Trends Cell Biol., 9:125-128, 1999.
Miyashita and Reed, Cell, 80:293-299, 1995.
Moore et al., Cancer Res., 58:5239-5247, 1998.
Mori et al., Cancer Res., 54(13):3396-3397, 1994.
Moriguchi et al., J. Biol. Chem., 270:12969, 1995.
Negherbohn, In: Handbook of Toxicology, Saunders (Ed.), PA, 1959.
Nobori et al., Nature, 368(6473):753-756, 1994.
Okamoto et al., Proc. Natl. Acad. Sci. USA, 1(23):11045-11049, 1994.
Orlow et al., Cancer Res., 54(11):2848-2851, 1994.
Papkoff et al., Mol. Cell. Biol., 14:463, 1994.
Phillips et al., Cancer, 83:41-47, 1998.
Reddel et al., Cancer Res., 48:1904-1909, 1988.
Remington's Pharmaceutical Sciences, 15th ed., 33:624-652, Mack Publishing Company, Easton, Pa., 1980.
Robinson et al., Curr. Opin. Cell Biol., 9:180-186, 1997.
Rodriguez-Viciana et al., Cell, 89:457-467, 1997.
Rowlands and Casida, The Toxicol., 36:235, 1997.
Serrano et al., Nature, 366:704-707, 1993.
Serrano et al., Science, 267(5195):249-252, 1995.
Shayesteh et al., Nat. Genet., 21:99-102, 1999.
Sibilia et al., Cell, 102:211-220, 2000.
Strange et al., Micro. Res. Techl., 52:171-181, 2001.
Sun et al., J. Cell Physiol., 173:279-284, 1997.
Toker et al., Nature, 387-673-676, 1997.
Tsujimoto and Croce, Proc. Natl. Acad. Sci. USA, 83(14):5214-5218, 1986.
Tsujimoto et al., Science, 228(4706):1440-1443, 1985.
Udeani et al. i, Cancer Res., 57:3424-3428, 1997.
Vanhaesbrock et al., Trends Biochem. Sci., 22:267-272, 1997.
Watenberg, In: Cancer Chemoprevention, CRC Press, Inc., Boca Raton, Fla., 19-39, 1992.
Weinberg, Science, 254(5035):1138-1146, 1991.
Wood et al., Pestic. Biochem. Physiol., 54:133-145, 1996.
Yano et al., Nature, 396:584-587, 1998.

1. A method of inhibiting growth in a lung cancer cell comprising contacting the cell with a therapeutically effective amount of deguelin or a derivative thereof in combination with a second agent.

2. The method of claim 1, wherein inhibiting comprises inducing apoptosis in the lung cancer cell.

3. The method of claim 1, wherein the second agent is a PI3K, MAPK or JNK inhibitor.

4. The method of claim 1, wherein the second agent is a chemotherapeutic agent.

5. The method of claim 4, wherein the chemotherapeutic agent is taxol or doxorubicin.

6. The method of claim 1, wherein the second agent is a radiotherapeutic agent.

7. The method of claim 1, wherein the deguelin derivative is 6a,2a-dehydrorotenone.

8. The method of claim 1, wherein the deguelin derivative is methoxyrot-2′-enoic acid.

9. The method of claim 1; wherein the deguelin derivative is tephrosin.

10. The method of claim 1, wherein the deguelin derivative is 7S-hydroxydeguelin.

11. The method of claim 1, wherein the deguelin derivative is rotenone.

12. The method of claim 1, wherein the deguelin derivative is 7a,13a-dehydrodeguelin.

13. The method of claim 1, wherein the deguelin derivative is 12-hydroxyrotenone.

14. The method of claim 1, wherein the deguelin derivative is 12,12a-dehydrorotenone.

15. The method of claim 1, wherein the deguelin derivative is isorotenone.

16. The method of claim 1, wherein the deguelin derivative is 4-chlororot-2′-enoic acid.

17. The method of claim 1, wherein the deguelin derivative is 1,2-dihydrodeguelin.

18. The method of claim 1, wherein the deguelin derivative is 2-phenylselenyl-1,2-dihydrodeguelin.

19. The method of claim 1, wherein the deguelin derivative is bromorot-2′-enoic acid.

20. The method of claim 1, wherein the cancer cell is a cell culture.

21. The method of claim 1, wherein the cancer cell is a tissue culture.

22. The method of claim 1, wherein the cancer cell is in a mammal.

23. The method of claim 22, wherein the mammal is a human.

24. The method of claim 1, wherein the cancer cell is a premalignant cancer cell.

25. The method of claim 1, wherein the cancer cell is a malignant cancer cell.

26. The method of claim 1, wherein the cancer cell is a metastatic cancer cell.

27. The method of claim 1, wherein the cancer cell is a non-small cell lung cancer cell, a small cell lung cancer cell, or a rare lung cancer cell.

28. The method of claim 27, wherein the non-small cell lung cancer is a squamous cell carcinoma, an adenocarcinoma or a large cell carcinoma.

29. The method of claim 27, wherein the small cell lung cancer is a lymphocytic small cell lung cancer, a intermediate small cell lung cancer or a combined small cell lung cancer.

30. The method of claim 29, wherein combined small cell lung cancer further comprises small cell lung cancer and squamous cell carcinoma.

31. The method of claim 29, wherein combined small cell lung cancer further comprises small cell lung cancer and adenocarcinoma.

32. The method of claim 27, wherein a rare lung cancer cell is a adenoid cystic carcinoma, a mesothelioma, a hamartoma, a lymphoma or a sarcoma.

33. The method of claim 27, wherein the lung cancer cell is a carcinoid tumor cell.

34. A method for treating or preventing lung cancer in a subject comprising providing to the subject a therapeutically effective amount of deguelin or derivative thereof in combination with a second agent.

35. The method of claim 34, further comprising inducing apoptosis in the cancer cell.

36. The method of claim 34, wherein the second agent is a PI3K, MAPK or JNK inhibitor.

37. The method of claim 34, wherein the second agent is a chemotherapeutic agent.

38. The method of claim 37, wherein the chemotherapeutic agent is taxol or doxorubicin.

39. The method of claim 34, wherein the second agent is a radiotherapeutic agent.

40. The method of claim 34, wherein the deguelin derivative is 6a,2a-dehydrorotenone.

41. The method of claim 34, wherein the deguelin derivative is methoxyrot-2′-enoic acid.

42. The method of claim 34, wherein the deguelin derivative is tephrosin.

43. The method of claim 34, wherein the deguelin derivative is 7S-hydroxydeguelin.

44. The method of claim 34, wherein the deguelin derivative is rotenone.

45. The method of claim 34, wherein the deguelin derivative is 7a,13a-dehydrodeguelin.

46. The method of claim 34, wherein the deguelin derivative is 12-hydroxyrotenone.

47. The method of claim 34, wherein the deguelin derivative is 12,12a-dehydrorotenone.

48. The method of claim 34, wherein the deguelin derivative is isorotenone.

49. The method of claim 34, wherein the deguelin derivative is 4-chlororot-2′-enoic acid.

50. The method of claim 34, wherein the deguelin derivative is 1,2-dihydrodeguelin.

51. The method of claim 34, wherein the deguelin derivative is 2-phenylselenyl-1,2-dihydrodeguelin.

52. The method of claim 34, wherein the deguelin derivative is bromorot-2′-enoic acid.

53. The method of claim 34, wherein the cancer is a premalignant cancer.

54. The method of claim 34, wherein the cancer is a malignant cancer.

55. The method of claim 34, wherein the cancer is a metastatic cancer.

56. The method of claim 34, wherein the cancer is a non-small cell lung cancer, a small cell lung cancer, or a rare lung cancer cell.

57. The method of claim 56, wherein the non-small cell lung cancer is a squamous cell carcinoma, an adenocarcinoma or a large cell carcinoma.

58. The method of claim 56, wherein the small cell lung cancer is a lymphocytic small cell lung cancer, a intermediate small cell lung cancer or a combined small cell lung cancer.

59. The method of claim 58, wherein combined small cell lung cancer further comprises small cell lung cancer and squamous cell carcinoma.

60. The method of claim 58, wherein combined small cell lung cancer further comprises small cell lung cancer and adenocarcinoma.

61. The method of claim 56, wherein the rare lung cancer is a adenoid cystic carcinoma, a mesothelioma, a hamartoma, a lymphoma or a sarcoma.

62. The method of claim 56, wherein the lung cancer is a carcinoid tumor.

63. The method of claim 34, wherein deguelin is provided to the subject before the second agent.

64. The method of claim 34, wherein deguelin is provided to the subject after the second agent.

65. The method of claim 34, wherein deguelin is provided to the subject at the same time as the second agent.

66. The method of claim 34, wherein deguelin is provided once.

67. The method of claim 34, wherein deguelin is provided more than once.

68. The method of claim 34, wherein the second agent is provided once.

69. Then method of claim 34, wherein the second agent is provided more than once.

70. The method of claim 34, wherein deguelin in combination with a second agent is provided once.

71. The method of claim 34, wherein deguelin in combination with a second agent is provided more than once.

72. The method of claim 34, wherein deguelin and the second agent are provided to a subject intratumoraly, intravenously, intraperitoneally, intramuscularly, orally, or by inhalation.

73. The method of claim 34, further comprising photodynamic therapy, or surgery.

74. A method for assaying for the inhibition of lung cancer cell growth comprising: a) providing a lung cancer cell sample; b) contacting the cell with an effective amount of deguelin or derivative thereof and a second agent; c) analyzing the cell for growth inhibition; and, d) comparing the inhibition of the cell growth in step (c) with the inhibition of a lung cancer cell in the absence of deguelin or derivative thereof and a second agent, wherein the difference in growth inhibition represents the growth inhibitory effect of deguelin or derivative thereof and a second agent.

75. The method of claim 74, wherein growth inhibition is analyzed by MTT assay.

76. The method of claim 74, further comprising analyzing the sample for induction of apoptosis.

77. The method of claim 76, wherein induction of apoptosis is analyzed by FACS.

78. The method of claim 74, further comprising analyzing the sample for inhibition of Akt activity.

79. The method of claim 78, wherein inhibition of Akt activity is analyzed by PI3K assay.

80. The method of claim 74, wherein the second agent is a PI3K, MAPK or JNK inhibitor.

81. The method of claim 74, wherein the second agent is a chemotherapeutic agent.

82. The method of claim 81, wherein the chemotherapeutic agent is taxol or doxorubicin.

83. The method of claim 74, wherein the second agent is a radiotherapeutic agent.

84. The method of claim 74, wherein the deguelin derivative is 6a,2a-dehydrorotenone.

85. The method of claim 74, wherein the deguelin derivative is methoxyrot-2′-enoic acid.

86. The method of claim 74, wherein the deguelin derivative is tephrosin.

87. The method of claim 74, wherein the deguelin derivative is 7S-hydroxydeguelin.

88. The method of claim 74, wherein the deguelin derivative is rotenone.

89. The method of claim 74, wherein the deguelin derivative is 7a,13a-dehydrodeguelin.

90. The method of claim 74, wherein the deguelin derivative is 12-hydroxyrotenone.

91. The method of claim 74, wherein the deguelin derivative is 12,12a-dehydrorotenone.

92. The method of claim 74, wherein the deguelin derivative is isorotenone.

93. The method of claim 74, wherein the deguelin derivative is 4-chlororot-2′-enoic acid.

94. The method of claim 74, wherein the deguelin derivative is 1,2-dihydrodeguelin.

95. The method of claim 74, wherein the deguelin derivative is 2-phenylselenyl-1,2-dihydrodeguelin.

96. The method of claim 74, wherein the deguelin derivative is bromorot-2′-enoic acid.

97. The method of claim 74, wherein the cancer sample is a non-small cell lung cancer, a small cell lung cancer, or a rare lung cancer.

98. The method of claim 97, wherein the non-small cell lung cancer is a squamous cell carcinoma, an adenocarcinoma or a large cell carcinoma.

99. The method of claim 97, wherein the small cell lung cancer is a lymphocytic small cell lung cancer, a intermediate small cell lung cancer or a combined small cell lung cancer.

100. The method of claim 99, wherein combined small cell lung cancer further comprises small cell lung cancer and squamous cell carcinoma.

101. The method of claim 99, wherein combined small cell lung cancer further comprises small cell lung cancer and adenocarcinoma.

102. The method of claim 97, wherein the rare lung cancer is a adenoid cystic carcinoma, a mesothelioma, a hamartoma, a lymphoma or a sarcoma.

103. The method of claim 97, wherein the lung cancer is a carcinoid tumor cell.

104. A pharmaceutical composition comprising deguelin or derivative thereof and a second agent.

105. The pharmaceutical composition of claim 104, wherein the second agent is a PI3K, MAPK or JNK inhibitor.

106. The pharmaceutical composition of claim 104, wherein the second agent is a chemotherapeutic agent.

107. The pharmaceutical composition of claim 106, wherein the chemotherapeutic agent is taxol or doxorubicin.

108. The pharmaceutical composition of claim 104, wherein the second agent is a radiotherapeutic agent.

109. The pharmaceutical composition of claim 104, wherein the deguelin derivative is 6a,2a-dehydrorotenone.

110. The pharmaceutical composition of claim 104, wherein the deguelin derivative is methoxyrot-2′-enoic acid.

111. The pharmaceutical composition of claim 104, wherein the deguelin derivative is tephrosin.

112. The pharmaceutical composition of claim 104, wherein the deguelin derivative is 7S-hydroxydeguelin.

113. The pharmaceutical composition of claim 104, wherein the deguelin derivative is rotenone.

114. The pharmaceutical composition of claim 104, wherein the deguelin derivative is 7a,13a-dehydrodeguelin.

115. The pharmaceutical composition of claim 104, wherein the deguelin derivative is 12-hydroxyrotenone.

116. The pharmaceutical composition of claim 104, wherein the deguelin derivative is 12,12a-dehydrorotenone.

117. The pharmaceutical composition of claim 104, wherein the deguelin derivative is isorotenone.

118. The pharmaceutical composition of claim 104, wherein the deguelin derivative is 4-chlororot-2′-enoic acid.

119. The pharmaceutical composition of claim 104, wherein the deguelin derivative is 1,2-dihydrodeguelin.

120. The pharmaceutical composition of claim 104, wherein the deguelin derivative is 2-phenylselenyl-1,2-dihydrodeguelin.

121. The pharmaceutical composition of claim 104, wherein the deguelin derivative is bromorot-2′-enoic acid.
